(19) United States
(12) Patent Application Publication
(10) Pub. No.: US 2022/0189614 A1
(43) Pub. Date: Jun. 16, 2022

(54) SYSTEMS AND METHODS FOR MATCHING CONSUMERS WITH CARE PROVIDERS

(71) Applicant: Pearl Long Term Care Solutions Inc., Rehoboth Beach, DE (US)

(72) Inventors: Emille BRYANT, Reston, VA (US); Myisha GATSON, Rehoboth Beach, DE (US); Tyrone GRANDISON, Tukwila, WA (US); Rabia PIACENTINI, Savannah, GA (US)

(73) Assignee: Pearl Long Term Care Solutions Inc., Rehoboth Beach, DE (US)

(21) Appl. No.: 17/121,438

(22) Filed: Dec. 14, 2020

(51) Int. Cl. G16H 40/20; G16H 10/60

(52) U.S. Cl. CPC G16H 40/20; G16H 10/60

(57) ABSTRACT

A method for matching consumer to care providers includes transmitting a first request for provider information from a matching system; receiving the provider information; transmitting a second request for consumer application information from the matching system; receiving the consumer application information; generating a common application packet based on the consumer application information; storing the common application packet; transmitting a third request for consumer search criteria from the matching system; receiving the consumer search criteria; generating a user-selectable list of care providers based on the provider information that matches the consumer search criteria; transmitting the user-selectable list from the matching system; receiving a selection of one or more providers of the user-selectable list; retrieving the stored common application packet, generating a resident application tailored for each of the one or more care providers based on the retrieved common application packet, and automatically transmitting each resident application to a provider user device.

### FIELD OF THE DISCLOSURE

The present disclosure relates to systems and graphical users interfaces for matching consumers with care providers.

### BACKGROUND OF THE DISCLOSURE

The traditional process of searching available rooms and/or beds at health care facilities, in particular long-term care facilities, is a manual and time intensive process. Typically, a consumer or a representative of the consumer searching for available rooms has to complete various disjointed and most times cumbersome steps of the application process. For example, traditional processes require the consumer or consumer representative to visit a website to obtain preliminary information regarding possibly available rooms, make a phone call to confirm availability and verify price, visit the care facility in-person to view amenities and floor plans, visit a physician in-person to obtain an assessment of level of care for the consumer, and visit a notary in-person for obtaining notarized documents for completing the application process. Some facilities may even require applicants (consumers or consumer representatives) to fill out lengthy paperwork prior to receiving a quote for an available room. Having to coordinate such steps that include a combination of website visits, phone calls, in-person visits, and lengthy paperwork can be overwhelming and time-consuming for a consumer or consumer representative.

### SUMMARY OF THE DISCLOSURE

Applicants have discovered systems and methods that enable a streamlined process for searching for health care facilities and for verifying price information and room availability at the health care facilities. The system may be configured to receive room availability and price information in real-time from care providers and to receive search criteria from a consumer for searching the availability of rooms provided by the care providers. The search criteria may include a preferred location, consumer's care needs, and price information. If a consumer's care needs (such as a level of care for the consumer) is not provided, the streamlined process may generate a recommendation of a level of care using a scoring process based on information received from the consumer.

The streamlined process may be a two-sided user experience for matching consumers searching for an available room that meets a consumer's care need (or a patient's care need) and financial budget to providers looking to advertise available rooms and care services. A consumer may be a shopper (such as a person or patient seeking care for themselves). A consumer representative (such as a family member of the consumer seeking care, a caregiver, a guardian, a referral agent, or an estate planner) may represent the consumer and may use the system on behalf of the consumer. In some embodiments, a consumer representative or set of consumer representatives may access the system to help a consumer coordinate care. A provider may be a care facility or a representative of a care facility. The two-sided user experience may include a bidding platform configured to allow consumers to bid on available rooms, and care levels offered by the providers. The bid may be at market value, above market value, or below market value.

In some embodiments, the system may be configured to streamline a care facility's search and resident application process for the care facility. The system may be configured to provide a consumer or consumer representative a simplified consumer search process that allows the consumer or consumer representative to submit a resident application in a minimal number of steps. The minimal number of steps may include consumer selections via a consumer-based graphical user interface. The minimal number of steps may include steps associated with searching facilities, viewing facilities that have available rooms in real-time viewing pricing of facilities and services, automatically generating a resident application applicable to a plurality of facilities based on consumer input, automatically applying to selected facilities by transmitting the resident application for display at a provider-based graphical user interface, and automatically receiving notifications of a status of the resident application.

In some embodiments, to streamline the care facility's search and resident application and resident application process, the system may be configured to receive information and documentation for completing a facility's search and for generating a resident application. The information and documentation for completing a facility search and for generating a resident application may be received at a graphical user interface configured for a consumer. In some embodiments, to streamline the care facility search and resident application process, the system may be configured to receive room availability, pricing, and details for listing available rooms and or units at one or more care facilities and receive resident applications at a graphical user interface configured for a provider.

In some embodiments, the resident application may be a common application applicable for applying to available rooms at many facilities. The common application enables an efficient booking process for both the consumer and the provider. The system may be configured to store common application data until the system receives a user selection to send the common application data to a selected care provider. For example, a consumer may submit consumer application information to the system, the system may be configured to receive the consumer application information and generate a common application for all the facilities that the consumer is interested in. Once generated, the common application may be stored in the system until the consumer makes a selection to submit the common application to one or more care providers. The system may request search criteria from the consumer and display a list of care providers that have rooms available that satisfy the search criteria. The consumer may then click a user-selectable icon of the system to select a care provider from the displayed list of care providers to view room availability and care provider information. In response to receiving the user selection, the system may be configured to display care provider details and associated information regarding room availability. Should the consumer decide to apply to a select care provider, the user may select to book or bid on the room offered by the care provider. In response to receiving the user selection to apply to the selected care provider, the system may be configured to transmit a stored common application profile that is based on the common application data to the care provider. The common application data may include collected demographic information and documents required for the resident intake process and allow the consumer to use the uploaded information to apply to multiple care providers. A graphical user interface configured for the provider may be configured to receive and display the transmitted common application.

In some embodiments, the consumer application information may be received via a graphical user interface configured for a consumer. In some embodiments, the consumer application information may include details that care providers associated with the system typically request from consumers interested in becoming a resident at their provider facility. The consumer application information may include consumer demographic information and elements of their search criteria. In some embodiments, the consumer application information may include consumer health information and a level of care applicable for the consumer.

In some embodiments, common application data may be generated by the system based at least on the received consumer application information. In some embodiments, the common application data may be stored in a centralized database. In some embodiments, the consumer graphical user interface may be configured to receive consumer search criteria. Based on the received consumer search criteria, the consumer graphical user interface may be configured to display a list of care providers that match the consumer search criteria. The consumer may select one or more of the care providers displayed to submit an application for residency. Upon selection of one or more care providers by the consumer, the system may be configured to determine what part (if not all) of the stored common application data is applicable to the selected one or more care providers. One or more resident applications may be generated based on the stored common application data applicable to the one or more care providers. In some embodiments, a plurality of different resident applications may be generated based on the stored common application data upon a consumer's selection (via a graphical user interface configured for a consumer) to submit resident applications to a plurality of care providers. Each resident application may be generated to include at least part of the stored common application data that is determined to satisfy applicant information associated with application requirements of a care provider that the consumer has selected for submitting a resident application.

In some embodiments, upon selection by a consumer to submit an application to one or more care providers, the stored common application data may be retrieved, and a resident application tailored for each of the one or more care providers may be generated based on the retrieved common application data. For example, upon selection (via a graphical user interface configured for a consumer) by a consumer to apply to a first care provider and a second care provider, the system may be configured to determine application requirements associated with the first care provider and the second care provider, collect first application information from the stored common application data that is determined to satisfy application requirements associated with the first care provider, collect second application information from the stored common application data that is determined to satisfy application requirements associated with the second care provider, generate a first resident application tailored for the first care provider based on the first application information of the stored common application data, and generate a second resident application tailored for the second care provider based on the second resident application information of the stored common application data. In some embodiments, the first application information and the second application information may be the same. In some embodiments, the first application information and the second application information may be different. In some embodiments, the generated first resident application may be automatically transmitted for display on a graphical user interface configured for a provider, and the first resident application may be accessible by the first care provider via the graphical user interface configured for a provider. The generated second resident application may be automatically transmitted for display on a graphical user interface configured for a provider, and the second resident application may be accessible by the second care provider via the graphical user interface configured for a provider. In some embodiments, the generated resident application based on the common application data may include, for example, one or more of: resident information that may include resident name and emergency contact; medical history that may include insurance information, physical details, results and/or records from medical examinations, Medicaid eligibility, and veteran's benefits; financials that may include financial disclosures, financial contact, financial application, and asset disclosures; a level of care assessment; PPD skin test (Tuberculosis) results; proof of insurance; power of attorney; advance directives; and guardianship documents.

In some embodiments, the system may be configured to save consumers money on long-term and short-term rehabilitative or respite care services, increase care facility occupancy, and increase revenues and profit for care facilities. In some embodiments, the system may include a web-based bidding platform for facility rooms and services, where providers opt in to using the platform.

In some embodiments, the system may include a graphical user interface configured to enable consumers to: search, select, and bid on unoccupied long-term or short-term rehabilitative and respite care units that are either currently unoccupied or will be unoccupied in the requested period; search for long-term or short-term rehabilitative and respite care facilities based on specified criteria which may include location, date, care needs, and specified price range; review available facility listings that meet the specified criteria; review facility details, unit and or room details, and ratings; submit a resident application for a room and or unit at a selected facility for review by the selected facility; receive notifications regarding a status of the resident application; and download floorplans of the room and or unit, conduct virtual tours and virtually meet the caregivers at the facility.

In some embodiments, the system may include a graphical user interface configured to enable providers to: create a listing of long-term or short-term care units, which are either currently unoccupied or will be unoccupied in the requested period, with searchable details such as location, date, floor plans, care services, and price range; review resident applications received from consumers for listings that meet the consumers' searchable specified criteria which may include location, date, care needs, and price range; accept or reject a resident application; accept, reject, or counter a price bid received as part of a resident application; and send notifications regarding a status of the resident application.

The graphical user interface described herein may be displayed on user devices. For example, the graphical user interface configured for a consumer may be displayed on a user device configured for the consumer. The graphical user interface configured for a provider may be displayed on a user device configured for the provider. According to some embodiments, a user device may display either the graphical user interface configured for the consumer or the graphical user interface configured for the provider based on a login into the system.

According to some embodiments, the system may be a matching system communicatively connected to one or more user devices. The system may be configured to transmit one or more requests for provider information and one or more requests for consumer information. The system may be configured to receive provider information from a provider via a provider user device and receive consumer information from a consumer via a consumer user device. The system may be configured to generate and store a common application packet (or common application data) based at least on the consumer information. The system may be configured to transmit a request for consumer search criteria and upon receipt of consumer search criteria, generate and transmit a user-selectable list of care providers that match the consumer search criteria and the consumer information. The system may be configured to receive a selection of one or more care providers from the user-selectable list, retrieve the stored common application packet, generate a resident application based on the common application packet, and automatically transmit the resident application to a corresponding provider user device. The system may be configured to generate a recommended level of care for a consumer based on the consumer information.

In some embodiments, a method for recommending a level of care for consumers includes: transmitting a first request for consumer information from the matching system to a consumer user device, the consumer information comprising a plurality of independency levels of a consumer, each independency level of the plurality of independency levels represents the consumer's ability to complete an activity of a plurality of activities, each independency level is associated with a designated point value; receiving the consumer information from the consumer user device; determining whether one or more of the designated point values associated with independency levels that represent the consumer's ability to complete one or more first activities of the plurality of activities lie within a threshold range; calculating a mathematical result based on the one or more designated point values and whether the one or more of the designated point values lie within the threshold range; generating a recommended level based on the mathematical result; and transmitting the recommended level of care to the consumer user device.

In any of these embodiments, the method may include transmitting a second request for provider information from the matching system to a provider user device; receiving the provider information from the provider user device; transmitting a third request for search criteria from the matching system to the consumer user device; and receiving the search criteria from the consumer user device.

In any of these embodiments, the method may include generating a user-selectable list of care providers based on the provider information that match search criteria and offers the recommended level of care and has availability during the user selected start date of care; transmitting the user-selectable list from the matching system to the consumer user device; receiving a selection of one or more care providers from the user-selectable list; and in response to receiving the selection, automatically providing booking options for a consumer stay at a residence of the one or more care providers.

In any of these embodiments, the search criteria may include a price preference for one or more available rooms at the residence of the one or more care providers, and the available rooms have a minimum price set by the one or more care providers, and wherein the price preference equals or exceeds the minimum price.

In any of these embodiments, the one or more activities may be associated with memory care or with non-memory care.

In any of these embodiments, the recommended level of care may be based on a plurality of care options that include one or more of memory care, independent living, assisted living, and skilled nursing.

In some embodiments, a method for matching consumers to care providers includes: transmitting a first request for provider information from a matching system to a provider user device; receiving the provider information from the provider user device; transmitting a second request for search criteria from the matching system to a consumer user device, the search criteria comprising at least one or more of a preferred location, price information, and consumer care needs; receiving the search criteria from the consumer user device; in response to receiving the search criteria, determining a first set of coordinates based on the search criteria; identifying first care providers based on the provider information that have available rooms that satisfy the search criteria that are located in a first area associated with the first set of coordinates.

In any of these embodiments, the method may include counting an amount of the first care providers; if the amount of first care providers is at least one, determining whether the amount of the first care providers is equal to, above, or less than a minimum amount; and if it is determined that the amount of the first care providers is equal to or above the minimum amount, automatically transmitting the first care providers as part of a list of care providers from the matching system to the consumer user device.

In any of these embodiments, wherein if the amount of first care providers is not at least one, the method may include updating the first set of coordinates to equal a second set of coordinates, the second set of coordinates having a second area that is larger than the first area by a first percentage.

In any of these embodiments, the method wherein if it is determined that the amount of first care providers is less than the minimum amount, the method may include identifying second care providers that have available rooms that satisfy the search criteria and that are located in a third area associated with a third set of coordinates, the third area having a value larger than the second area by a second percentage, counting an amount of the second care providers, and updating the first set of coordinates to equal the third set of coordinates if the amount of second care providers is greater than the amount of first care providers by over a third percentage.

In any of these embodiments, wherein if the amount of second care providers is not greater than the amount of first care providers by over the third percentage, the method may include detecting a center of a grouping of the second care providers, the center having center coordinates, setting the center coordinates as a center of a fourth area, the fourth area associated with a fourth set of coordinates, and identifying fourth care providers that have available rooms that satisfy the search criteria and that are located in the fourth area.

In any of these embodiments, wherein if an amount of fourth care providers is greater than the amount of third providers by over a fourth percentage, the method may include updating the first set of coordinates to equal the fourth set of coordinates.

In any of these embodiments, the fourth percentage may equal the third percentage and the second percentage may equal the first percentage.

In any of these embodiments, wherein if an amount of fourth care providers is not greater than the amount of third providers by over a fourth percentage, the method may include transmitting the third care providers as part of the list of care providers from the matching system to the consumer user device.

In some embodiments, a method for matching consumers to care providers includes: transmitting a first request for provider information from a matching system to a provider user device; receiving the provider information from the provider user device; transmitting a second request for consumer application information from the matching system to a consumer user device; receiving the consumer application information from the consumer user device; in response to receiving the consumer information, generating a common application packet based on the consumer application information; storing the common application packet; transmitting a third request for consumer search criteria from the matching system to the consumer user device; receiving the consumer search criteria from the consumer user device; generating a user-selectable list of care providers based on the provider information that matches the consumer search criteria; transmitting the user-selectable list of care providers from the matching system to the consumer user device; receiving a selection of one or more care providers from the user-selectable list; and in response to receiving the selection, retrieving the stored common application packet, generating a resident application tailored for each of the one or more care providers based on the retrieved common application packet, and automatically transmitting each resident application to the provider user device.

In any of these embodiments, each resident application may be accessible to a care provider of the one or more care providers associated with each resident application via the provider user device.

In any of these embodiments, the consumer application information may include one or more of demographic information, consumer health information, and a level of care applicable for the consumer.

In some embodiments, a system for matching consumers to care providers includes: a first user device configured for registered providers; a second user device configured for consumers; a third user device; a matching system configured to communicatively connected the first user device, the second user device, and the third user device, the matching system comprising one or more programs that include instructions to: transmit a first request for provider information to the first user device; receive the provider information from the first user device; transmit a second request for consumer application information to the second user device; receive the consumer application information from the second user device; in response to receiving the consumer information, generate a common application packet based on the consumer application information; store the common application packet; transmit a third request for consumer search criteria from the matching system to the second user device; receive the consumer search criteria from the second user device; generate a user-selectable list of care providers based on the provider information that matches the consumer search criteria; transmit the user-selectable list of care providers to the second user device; receive a selection of one or more care providers from the user-selectable list; and in response to receiving the selection, retrieve the stored common application packet, generate a resident application tailored for each of the one or more care providers based on the retrieved common application packet, and automatically transmit each resident application to the first user device.

In any of these embodiments, each resident application may be accessible to a care provider of the one or more care providers associated with each resident application via the first user device, and the one or more programs of the matching system may include instructions to transmit administrative information associated with one or more resident applications from the matching system to the third user device and to receive one or more administrative updates from the third user device based on the administrative information.

In any of these embodiments, the consumer application information may include one or more of demographic information, consumer health information, and a level of care applicable for the consumer.

In addition, it is also to be understood that the singular forms “a”, “an,” and “the” used in the following description are intended to include the plural forms as well, unless the context clearly indicates otherwise. It is also to be understood that the term “and/or,” as used herein, refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units, but do not preclude the presence or addition of one or more other features, integers, steps, operations, elements, components, units, and/or groups thereof.

In the following description of the disclosure and embodiments, reference is made to the accompanying drawings in which are shown, by way of illustration, specific embodiments that can be practiced. It is to be understood that other embodiments and examples can be practiced, and changes can be made, without departing from the scope of the disclosure.

### BRIEF DESCRIPTION OF THE DRAWINGS

The invention will now be described, by way of example only, with reference to the accompanying drawings, in which:

FIG. 1 shows an exemplary system for matching users to care providers, according to some embodiments.

FIG. 2 shows an exemplary landing page of a system, according to some embodiments.

FIG. 3 shows an exemplary subscriber advertisement displayed on a landing page, according to some embodiments.

FIG. 4 shows an exemplary set of initial search criteria boxes for consumers to fill with information, according to some embodiments.

FIGS. 5A-5F show an exemplary results page, according to some embodiments.

FIG. 6 shows an example of an information page for a selected provider facility, according to some embodiments.

FIG. 7 shows an example of a room selection page, according to some embodiments.

FIG. 8 shows an example of a level of care selection page, according to some embodiments.

FIG. 9 shows an example of an additional services page, according to some embodiments.

FIG. 10 shows an exemplary booking summary page, according to some embodiments.

FIG. 11 shows an exemplary booking confirmation page, according to some embodiments.

FIG. 12 shows an exemplary make an offer page for a bid, according to some embodiments.

FIG. 13 shows an example of an offer history, according to some embodiments.

FIG. 14 shows an example of a landing page for providers that may be displayed on a graphical user interface, according to some embodiments.

FIGS. 15A and 15B show examples of a listing page for providers, according to some embodiments.

FIGS. 16A-16H show examples of questions or statements associated with a listings page for creating an advertisement of an available room and or unit at a provider facility, according to some embodiments.

FIG. 17 shows an exemplary flowchart that describes a method for responsively re-calibrating search results returned to a consumer on a map.

FIGS. 18A-18C show an exemplary flowchart of a high level architecture of a communications system of a system, according to some embodiments.

FIG. 19 shows an example of an AWS infrastructure.

FIG. 20 shows an example of a service pod.

FIG. 21 shows an example of a sequence diagram of a user browser request, according to some embodiments.

FIG. 22 shows an example of a path of a request, according to some embodiments.

FIG. 23 shows a flowchart of an exemplary method for recommending a level of care for consumers, according to some embodiments.

FIG. 24 shows a flowchart of an exemplary method for matching consumers with care providers, according to some embodiments.

FIG. 25 illustrates a functional block diagram of a computing device, according to some embodiments.

FIGS. 26A-26B show a flowchart that describes an example method for consumers finding, bidding and booking care providers, according to some embodiments.

FIG. 27 shows a flowchart of an exemplary method for submitting a common application to care providers, according to some embodiments.

### DETAILED DESCRIPTION OF EXEMPLARY EMBODIMENTS

The systems and methods disclosed herein describe a two-sided system that may be configured to allow consumers to seamlessly search for services and accommodations for their short term rehabilitative and respite or long term care needs and allow providers to list and manage their listings of available rooms and services to be searched by consumers. The system may be configured to receive search criteria from a consumer (such as preferred location, consumer's care needs, and price information). If a level of care for the consumer is not received, the streamlined process may generate a recommendation for a level of care based on information received from the consumer and include the recommendation as part of the search criteria. The system may be configured to apply the search criteria to identify an amount of care providers that satisfy the consumer's search criteria. The identified care providers may be automatically displayed as part of a user-selectable list of care providers on a graphical user interface configured for the consumer. The graphical user interface may be displayed on a user device.

In some embodiments, the systems and methods described herein may include graphical user interfaces communicatively connected for displaying consumer information to care providers and for displaying care provider information to consumers on consumer user devices. In some embodiments, data submitted by a consumer or consumer representative via a first graphical user interface configured for a consumer may be displayed on a second graphical user interface configured for a care provider. Similarly, data submitted by a care provider via the second graphical user interface configured for a provider may be displayed on the first graphical user interface configured for a consumer. The first graphical user interface may be displayed on a first user device and the second graphical user interface may be displayed on a second user device.

In some embodiments, the first graphical user interface may be configured to provide price transparency of available rooms, view features and amenities associated with available rooms and units, submit booking and/or bids for available units and allow a consumer to populate a common resident application based on demographic and other consumer information. The system contains a centralized resource of user data that is used to generate resident application information; enabling providers to access this common application data (“common application”) whenever they please. It also reduces the cognitive load and stress for the user from repeatedly filling out the same information for each provider they need to interact with.

In some embodiments, upon receipt of the bid, the provider may review the price bid (if that engagement pathway is the one taken by the consumer) and send a notification to the consumer regarding if the price bid has been accepted, rejected, or if a counter offer has been proposed. In some embodiments, the second graphical user interface may be configured to receive the resident bid offer, automatically notify a care facility when a resident application has been received for a room located in their care facility, and send a notification regarding status of the resident application to the consumer or consumer representative via the first graphical user interface. In some embodiments, the available room may be unoccupied rooms and or units.

In some embodiments, the process of a consumer booking a stay at a care facility may be accomplished via the first graphical user interface configured for the consumer. The process of a consumer booking a stay may include searching available rooms at care facilities, viewing pricing, services, photos, and reviews associated with the care facilities, and applying (either a booking or bidding option) via a common application for an available room.

In some embodiments, the systems and methods described herein are configured to incorporate external resources into the common resident application process to reduce the number of manual steps for the consumer. For example, the systems and methods may be configured to determine if completion of the resident application requires external documentation such as notarized documentation or a health practitioner's care assessment. If it is determined that notarized documents are to be submitted as part of the resident application, the systems and methods may be configured to request a mobile notary on behalf of the consumer. If it is determined that a care assessment is to be submitted as part of the resident application, the systems and methods may be configured to conduct a preliminary level of care assessment, request a care assessment with a telehealth practitioner on behalf of the consumer, or both conduct a preliminary level of care assessment and request a care assessment with a (telehealth) practitioner on behalf of the consumer. In some embodiments, the external resources may be requested by or on behalf of the consumer based on inputs received by the consumer at the first graphical user interface.

In some embodiments, the first graphical user interface may be configured for displaying a sign up page for registering a consumer, displaying a list of residences having available rooms and or units for, allowing a consumer to search through the list of residences using a location, care level, ratings and price filters; displaying search results on a map and in the list of residences (provider facilities); updating the selected filters on the search results page and ordering the results based on location, care level offerings, ratings, or price, or based on a consumer's filter selections; displaying complete information about a residence; setting a residence as a saved residence to a list of favorites; creating a storage of saved residences; allowing a consumer to bid on or book a residence for one resident (applicant) at a time; allowing a resident to create, view, update, or delete a resident account via the first graphical user interface; allowing a resident to view a booking history, view a current status of an application, and cancel a booking via the resident account; receiving an intake application and required documentation, the intake application may be from a collection of documents stored on the platform that enable their reuse across applications for multiple providers (“the common application”); sending notifications on bid statuses and the booking process; sending notifications via email or text; limiting view of residences for user that are registered users of the system; logging in, securely and in a privacy-preserving manner, using biometric identifiers (in one embodiment, a cloud-based, homomorphic encryption service may be used to achieve this); allowing a consumer to add and/or manage members of their care team and family members listed in the system; communicating messages between the consumer and the provider by transmitting messages between the first graphical user interface and the second graphical user interface; allowing a resident to share residence information and provide a communication pathway for discussions about the shared residence with the care team and family members listed in the system; allowing a consumer to rate a shared residence; displaying additional services during a booking process of a room and displaying a total price that includes the unit base, care level price and the price for all selected additional services; scheduling an offline service with an offline provider via online calendar integration; displaying a comparison of two residences and see the results on the first graphical user interface; displaying documents for signing digitally, receiving a digital signature from the consumer, and transmitting the received digital signature to the second graphical user interface for displaying the received digital signature to the provider during the application process;

In some embodiments, the first graphical user interface may be configured for sending text confirmation for sign up and log in; displaying options to login to the system via social networks (such as Google, Twitter, Facebook); receiving and input to login a user via social networks; creating a best deals subscription based on selected filters (price, location, care level), and sending out notification of the best deals based on a selected notification frequency; searching for multiple residents at once (the amount of residents searchable may be limited based on aesthetics and ease of use); allowing a user to bid and book for multiple residents at once; displaying options for purchasing an insurance-like instrument and receiving a reimbursement; receiving input from the user for purchasing this insurance-like instrument; and displaying ratings and reviews and receiving submissions from a user for ratings and reviews. In some embodiments, the first graphical user interface may be configured to receive inputs associated with one or more items, pages, details, icons displayed on the first graphical user interface.

In some embodiments, the second graphical user interface may be configured for displaying a sign up page for registering a provider organization, creating an account for an organization; displaying residences to allow the provider to add/edit/delete a residence, displaying an information page to allow the provider to add general information, images, staff information; displaying listing of each residence to allow the provider to add/edit/remove a listing for each residence; displaying and receiving input from the user for managing the availability of units; displaying a dashboard with statistics associated with provider; displaying a status of received bids, and configured for receiving input from the user to accept the bid, decline the bid, make a counter offer; displaying the bidding information per room; displaying resident applications and configured for receiving input from the provider for approving the applications or declining the applications; displaying move-in information; displaying a list of all the transactions and details about them (resident, residence, date, status, amount); displaying additional services available for each room or residence and receiving input from the provider for managing the additional services (add/edit/delete services for each residence and set the monthly price for each service); receiving and transmitting messages between the consumer and provider via the first graphical user interface and the second graphical user interface.

In some embodiments, the second graphical user interface may be configured for displaying inquiries and alerts associated with an application or a resident; managing a user that is a part of a provider organization team (adding new members to the organization team and assigning them different roles and permissions); displaying revenue reports; displaying residents' reviews; viewing and receiving input from a user to reply to residents' reviews; and displaying, receiving, and transmitting messages within the system between a provider and an administrator of the system. In some embodiments, the second graphical user interface may be configured to receive inputs associated with one or more items, pages, details, icons displayed on the second graphical user interface.

In some embodiments, an administrative server may be accessible by an administrator of the system. In some embodiments, a third graphical user interface may be communicatively connected to the administrative server. The third graphical user interface may be configured for display on an administrator user device. The third graphical user interface may be configured for displaying and editing resident accounts (details about residents and their booking history), provider accounts (review and approve or decline them), listing (designate which listings to approve or block), transactions (receive payments, view details about transactions, search and filtering options), and notification history. In some embodiments, the third graphical user interface may be configured for offline services management conducted by the administrator. In some embodiments, the third graphical user interface may be configured for displaying and managing ratings and reviews; displaying, receiving, and transmitting messages with provider organizations; displaying a history of messages between providers and residents; and creating and displaying an approval flow for listing and bookings.

In some embodiments, a backend server may be configured to implement logic and application programming interface (API) methods to support the features of the first graphical user interface, the second graphical user interface, and the administrative server. In some embodiments, the backend server may include the administrative server. In some embodiments, the first graphical user interface and the second graphical user interface may be mobile responsive. In some embodiments, the backend server may be configured for integration with an electronic (cloud-based) notification service (such as Amazon SNS), a third party service for purchasing an insurance-like instrument, an electronic (cloud-based) telephonic service (such as Twilio Fax API), and LTC software for extracting info about room and unit availability. In some embodiments, the backend server may be configured to implement a scheduling service for prospective residents to be able to schedule services, in real life, with medical service providers, legal service providers, and financial service providers.

In some embodiments, the system may be configured to send notifications between a consumer and a provider. The notifications may be sent via communication medium, for example, via email, text, or in-platform collaboration tools. In some embodiments, the notifications may include updates on the best deals available. In some embodiments, the best deals notifications are available for registered and non-registered (aka “signed up”) users. A user may select the filters for the deals he/she wants to be notified about: location, care level(s), price range and the frequency of such notifications. The user may receive notifications for each deal that conforms to the selected filters.

In some embodiments, the notifications may include updates on the application process after booking. In some embodiments, the notifications on bids may be available for registered users only. If a user makes a bid, he/she then should receive email notifications on the status update of that bid—whether it has been declined or accepted by the provider or whether the provider has made a counter bid. The user may also receive reminders to make the booking if the bid has been accepted by the provider before an expiration time of the bid elapses.

In some embodiments, the notification may include updates on the booking process. In some embodiments, once a user has paid for the room, he/she may be notified about the next steps if he/she doesn't complete them right-away (such as complete the application, provide requested documents). The user may also be notified about the actions performed by the provider: e.g. accepted/declined the application, asked to provide additional documents.

In some embodiments, the notifications may include updates on the registration status of a care team member, family member, or friend. In some embodiments, when a user adds a new care team member, a family member or a friend, that added member receives an invitation to join as a registered user of the system. Once that person creates an account with the system, the resident or the care-giver may receive a notification saying that the person has joined as a registered user of the system in the role they specified. Once the person has joined as a registered user of the system, that person may help the resident or care-giver coordinate care at a care provider.

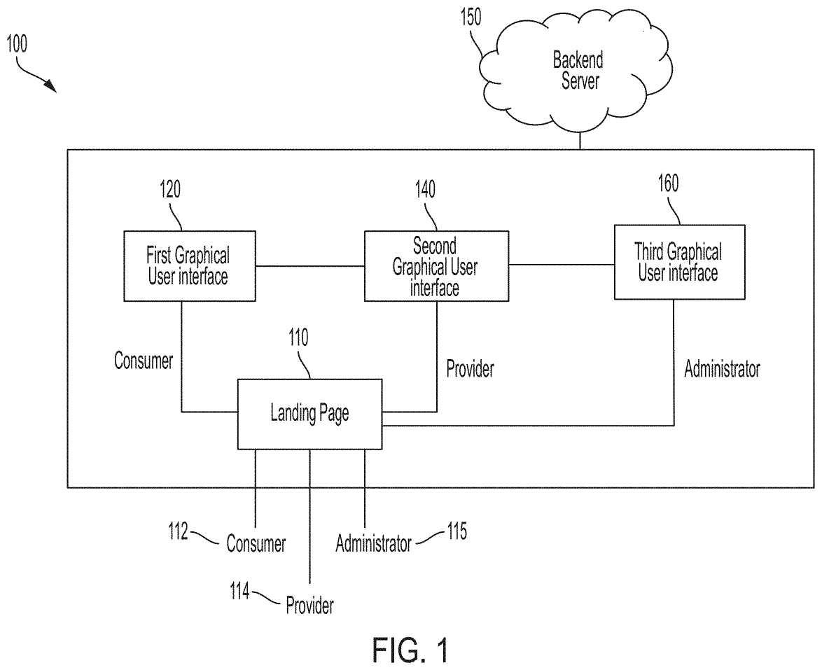

FIG. 1 shows an exemplary system 100 for matching users to care providers, according to some embodiments. The system 100 may include a landing page 110 configured to receive information from a user and classify the user as a consumer 112, a provider 114, or a system administrator 115 based on the information received on the landing page from the user. In some embodiments, the landing page may be a display on a graphical user interface. Based on inputs received at the landing page 110, a first graphical user interface 120, a second graphical user interface 140, or a third graphical user interface 160 may be displayed subsequent to the landing page 110. The system 100 may include a first graphical user interface 120 of the system 100 configured for receiving information from the consumer 112 and displaying information to the consumer 112. The system 100 may include a second graphical user interface 140 configured for receiving information from the provider 114 and displaying information to the provider 114. The system 100 may include a third graphical user interface 160 configured for receiving information from the system administrator 115 and displaying information to the administrator 115. The first graphical user interface 120 may be communicatively connected to the second graphical user interface 140. The first graphical user interface 120 may be accessed by one or more consumers 112. A consumer may be, for example, a person seeking care for themselves, a person seeking a room as a primary residence, a family member of the person seeking care for themselves, an estate planner researching options for their client, a referral agent using the system on behalf of their consumers, or a representative of the person seeking care for themselves. The second graphical user interface 140 may be accessed by one or more providers 114. A provider may be a representative of a long-term or short-term rehabilitation or respite care facility. For example, the representative may be a facility manager, a long-term care executive, executive administrators, chief financial officers, or a member of the sales and marketing team. The third graphical user interface 160 may be accessed by one or more system administrators. The first graphical user interface 120, the second graphical user interface 140, and the third graphical user interface 160 may be communicatively connectable to a backend server 150. The backend server may be accessible by a system administrator 115 for making data changes on behalf of consumers 112 or providers 114.

In another embodiment, there may be no landing page 110 and consumers 112 may access the first graphical user interface 120 directly, providers 114 may access the second graphical user interface 140 directly, and administrators 115 may access the third graphical user interface 160.

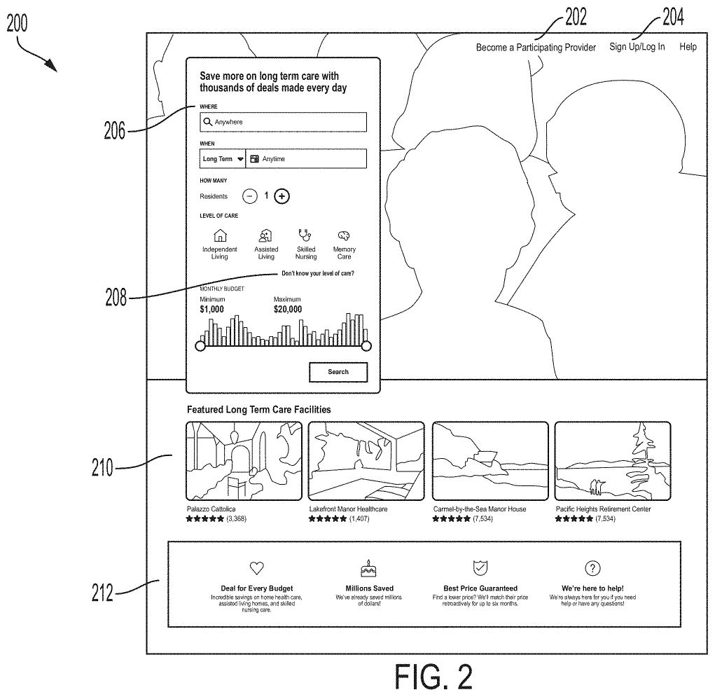

FIG. 2 shows an exemplary landing page 200 of a system, according to some embodiments. Landing page 200 may be an example of the landing page 110 or the first graphical user interface 120 of system 100. In some embodiments the landing page 200 may be a display of a landing graphical user interface. The landing page 200 may be configured to receive a request from the user to become a participating provider via a user-selectable icon 202. The landing page 200 may be configured to receive a request from the user to become a registered user of the system or to log into an account of a registered user of the system via a user-selectable icon 204. The landing page 200 may be configured to request and receive initial search information 206 from a user to refine the user's search for a long-term or short-term care facility. The initial search information 206 may include a preferred location of the facility, a preferred type of stay (short-term versus long-term) at the facility, a start date for the stay, a number of residents looking to stay at the facility, a resident's care need which may include the level of care suitable for the one or more residents looking to stay at the facility, and a preferred resident monthly price budget (or budget range) for paying for the room and services at the facility. The level of care may include one or more of independent living, assisted living, skilled nursing, memory care, respite care and short-term rehabilitative stays. The system may be configured to provide a preliminary level of care assessment should a user select a recommendation icon 208. In response to user selection of the recommendation icon 208, the system may be configured to determine a recommendation of a level of care (a preliminary level of care assessment) based on a user's response to a plurality of statements or questions.

In some embodiments, the preliminary level of care assessment may be based on functional assessment data collected from the consumer. The functional assessment data may include questions or statements associated with various daily or routine activities of daily living (ADLs) and Instrumental Activities of Daily Living (IADLs) for assessing a level of care suitable for the person seeking care. In some embodiments, the functional assessment data may include questions or statements for assessing an independency level of a consumer. For example, the functional assessment data may include questions or statements directed to activities such as bathing, dressing, toileting, transferring, eating/feeding, continence (bowel and bladder), walking, wheeling, stair climbing, mobility, meal preparation, housekeeping, laundry, money management, medication administrations, and memory care. Such functional assessment statements or questions may be associated with a functional assessment area to determine whether a consumer's level of care is categorized as memory care, independent living, assisted living, and skilled nursing. In some embodiments, a response or answer to each functional assessment statement or question may be either: a first response—which indicates that the applicant never requires assistance with this activity; a second response—which indicates applicant requires assistance fewer than a predetermined number of days per week or is only experiencing slight impairments completing the task; a third response—which indicates that the applicant is usually not able to independently complete this activity; and a fourth response—which indicates the applicant is never independent with this activity. In some embodiments, the first response may be “always,” the second response may be “usually independent,” the third response may be “usually not,” and a fourth response may be “never.”

In some embodiments, numerical points may be designated to each response received by the consumer. A recommendation for a level of care for the applicant may be based on mathematical manipulation of all the accumulated points and their evaluation against a continuum of thresholds. In some embodiments, a recommendation for a level of care for the applicant may be based on a calculation of an overall score based on the designated points. The overall score may be an average of the designated scores for each response.

In some embodiments, a first functional assessment area may be associated with memory care. In some embodiments, a second functional assessment area, a third functional assessment area, and a fourth functional assessment area may be associated with non-memory care. In some embodiments, the second functional assessment area may be associated with independent living. In some embodiments, the third functional assessment area may be associated with assisted living. In some embodiments, the fourth functional assessment area may be associated with skilled nursing.

In some embodiments, in response to submission of search criteria that may include consumer information and the recommended level of care, a list of available rooms and or units at one or more facilities that satisfy the consumer information and that offer the recommended level of care may be displayed at the first graphical user interface configured for a consumer. The list of available rooms may be based on received consumer information and the recommended level of care.

In some embodiments, the landing page 200 may include an advertisement 210 of featured facilities. The advertisement 210 may be based on the initial search information 206 received from the user. For example, the advertisement 210 may dynamically list featured facilities based on the preferred location entered as part of the initial search information 206. The featured facilities of the advertisement 210 may be a set from the top 30 facilities, a set from the top 20 facilities, or a set from the top 10 facilities. The advertisement may show a predetermined amount of featured facilities at one time so that the advertisement 210 of the predetermined amount of featured facilities displays a clear image of the featured facilities and legible rating information of the feature facilities.

In some embodiments, the landing page 200 may include an informational strip that includes user-selectable icons configured to display more information regarding an explanation of savings based on care level, how many users have saved by using the system, description of a best price guarantee of the system, and options to receive help using the system. The best price guarantee may guarantee to the user that through use of the system, the provider prices displayed on the system are comparable to other provider prices in that particular preferred location. The best price guarantee may guarantee to the user that through use of the system, the unit and/or room prices displayed on the system are comparable or less than any other room or unit in the particular facility.

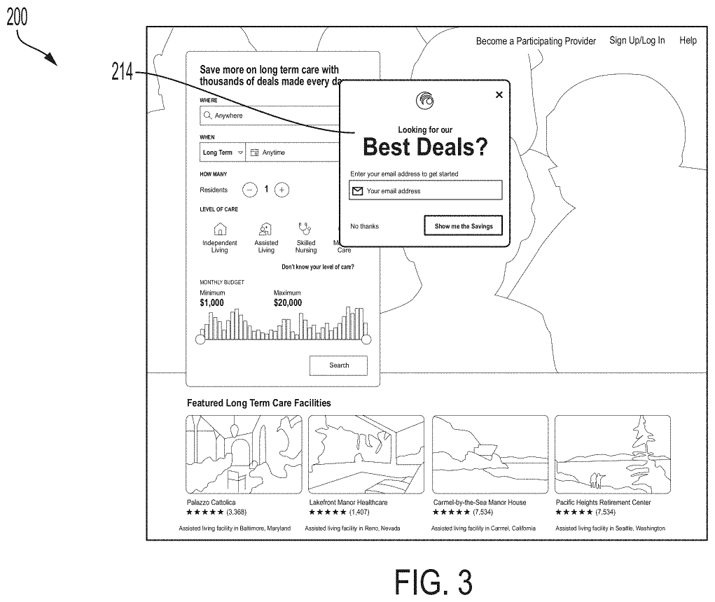

FIG. 3 shows an exemplary subscriber advertisement 214 displayed on the landing page 200, according to some embodiments. The subscriber advertisement 214 may be configured to enroll a person as a subscriber to the system. According to some embodiments, the subscriber advertisement 214 may be configured to request information to subscribe the user to the system for receiving updates on deals as they become available.

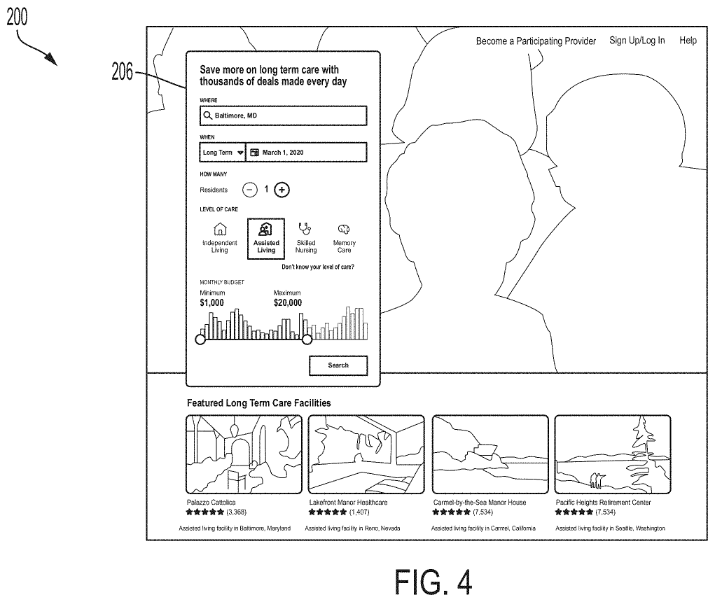

FIG. 4 shows an exemplary set of initial search information 206 that includes initial search criteria boxes for consumers to fill with information, according to some embodiments. In the example of FIG. 4, the preferred location is Baltimore, Md., the length of stay is long-term starting March 1^{st}, the stay is for one resident, the level of care is assisted living, and the monthly budget is set to a minimum of $1,000 and a maximum of $20,000. In some embodiments, a room and or unit can have one or multiple care levels associated with it. In some embodiments, multiple selection of care levels is available for independent living, assisted living, and skilled nursing. In some embodiments, memory care cannot be combined with other care levels. When searching for a room, a user can select multiple care levels including all four care levels above. Since current industry constraints dictate that memory care cannot be combined with independent living, assisted living, and skilled nursing, when all levels of care are selected for search purposes (in current embodiments), “AND” logic may be applied when defining the search results for independent living, assisted living, and skilled nursing, and “OR” logic may be applied to memory care when defining the search results as this care level cannot be combined with other care levels. In some embodiments, in response to submitting the initial search information 206 at the landing page 200, the system may be configured to display a results page configured for a consumer.

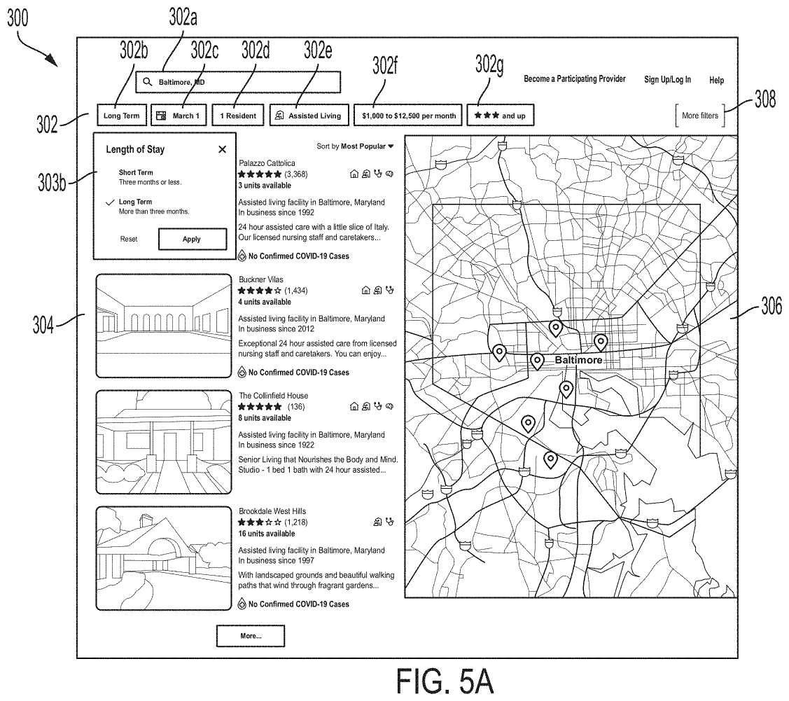

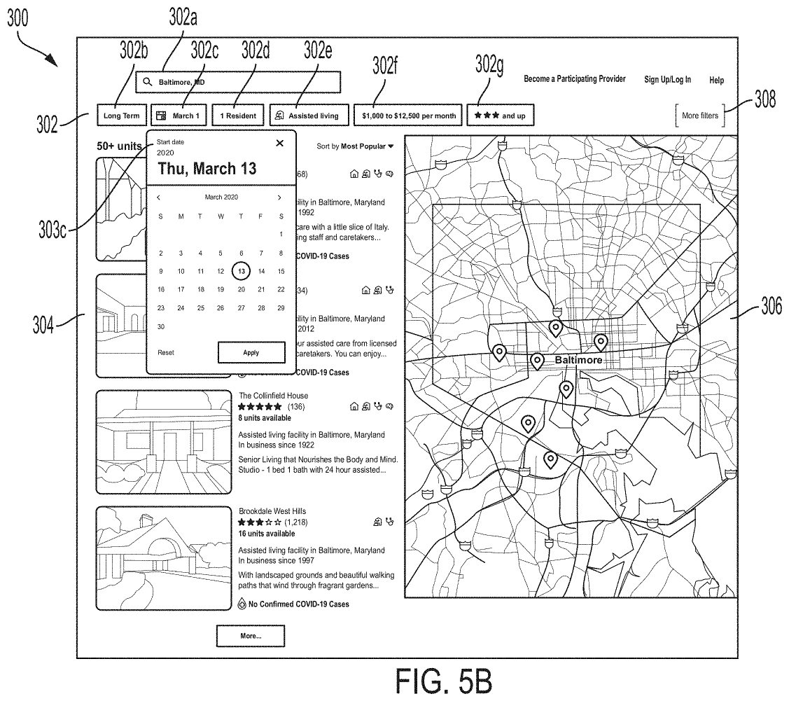

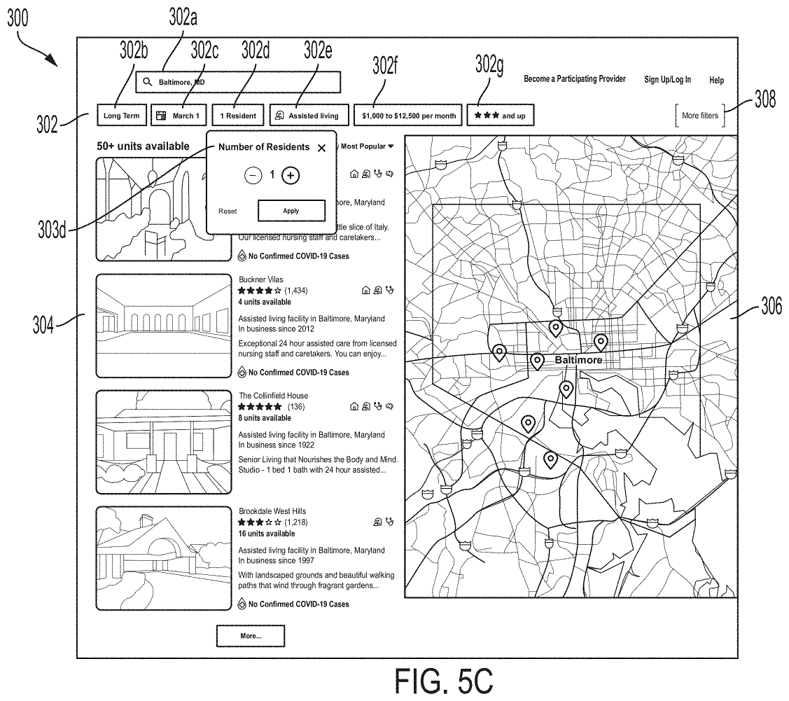

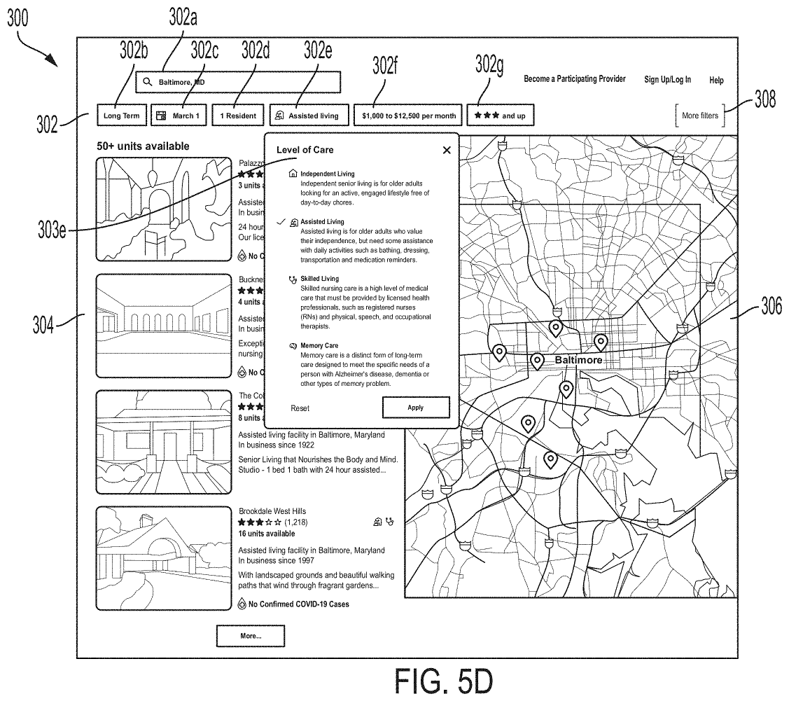

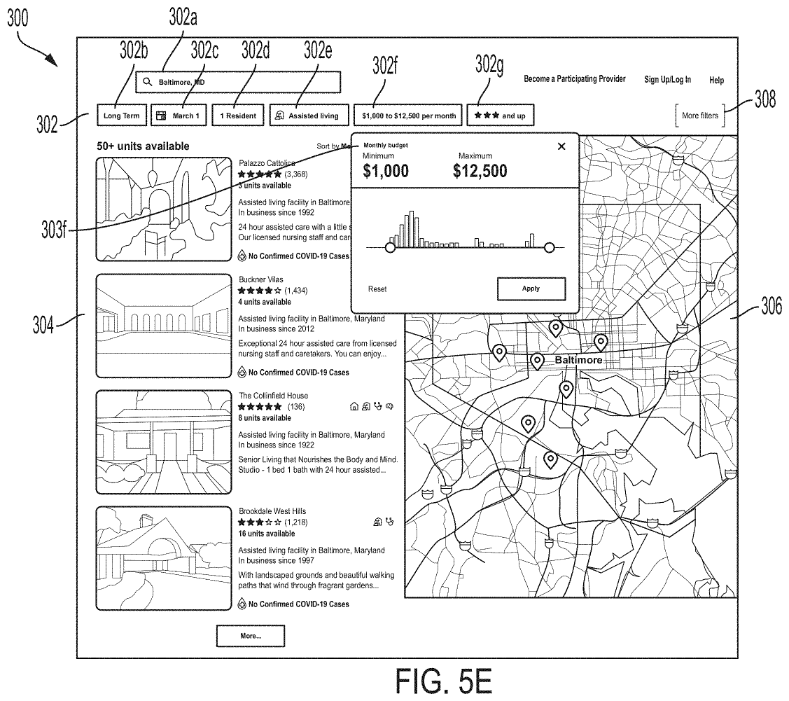

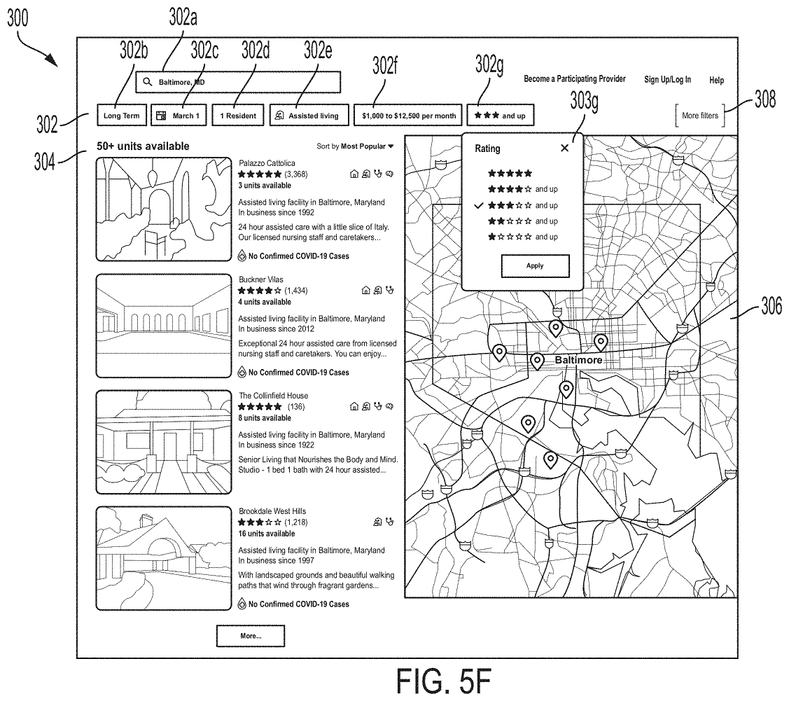

FIG. 5A shows an exemplary results page 300, according to some embodiments. The results page may be displayed on the first graphical user interface 120. The results page 300 may include a summary section 302 of the initial search information 206. Based on the initial search information, the results page 300 may be configured to display a list 304 of providers that have available rooms and or units that meet the criteria submitted as the initial search information 206 and display a corresponding map 306 of the providers that have available rooms and or units. The list 304 may be generated by the system based on the initial search information 206. Additional filters 308 that affect the list 304 and the corresponding map 306 may include a specification of whether a resident would like to share a room, if pets are allowed, and whether the consumer would like to implement a cut-off for showing providers that have a specific rating level. In some embodiments, the results page 300 may be displayed only if a registered user is logged into the system or if the user is not registered. In some embodiments, a limited version of the results page 300 may be displayed if a registered user is not logged into the system. In some embodiments, the limited version may include no more than a predetermined number of photos, no more than a predetermined number of listed amenities, and ratings with no more than a predetermined number of reviews. For example, the limited version may include no more than 3 photos of residence, lists 2 of the amenities, ratings with no more than 2 reviews, and link at the bottom urging registration and or signing in to find out more about this residence.

In some embodiments, the results page 300 may be configured to show a predetermined number of providers of a generated list 304 at a time. In the example of FIG. 5A, the results page 300 is configured to show a maximum number of listings of providers (from list 304) having available units at a time. The maximum number of listings may be configured to allow consumers a clear and legible view of the listings. The providers actively displayed may dynamically change in response to a user scrolling through the list 304. The list 304 may include one or more photos of the provider facility, name and location of the provider facility, a cumulative residence rating and an associated number of raters, an icon to indicate the level or levels of care available at the provider facility, a number of available units at the provider facility, how long the provider facility has been in business, and an incorporation date of the business. In some embodiments, the list 304 may include an indicator of a status associated with important indicators, such as COVID-19 status. For example, if applicable, the indicator may display that there are no confirmed COVID-19 cases at the provider facility. In some embodiments, a mouseover of a provider facility from the list 304 may highlight a location of the provider facility in the corresponding map 306.

In some embodiments, the summary section 302 may be dynamically updated to display the list 304 of providers with available units and the corresponding map 306 of the providers with available units. For example, the summary section 302 may be updated in response to user selection of a summary icons 302a-302g. Upon selection of any of the summary icons, the system may be configured to display additional options. An example of additional options of a length of stay associated with summary icon 302b is shown in FIG. 5A as display 303b. An example of additional options of applicable dates of stay associated with summary icon 302c is shown in FIG. 5B as display 303c. An example of additional options of a number of residents associated with summary icon 302d is shown in FIG. 5C as display 303d. An example of additional options of a level of care associated with summary icon 302e is shown in FIG. 5D as display 303e. An example of additional options of a price range associated with summary icon 302f is shown in FIG. 5E as display 303f. An example of additional options of ratings associated with summary icon 302g is shown in FIG. 5F as display 303g.

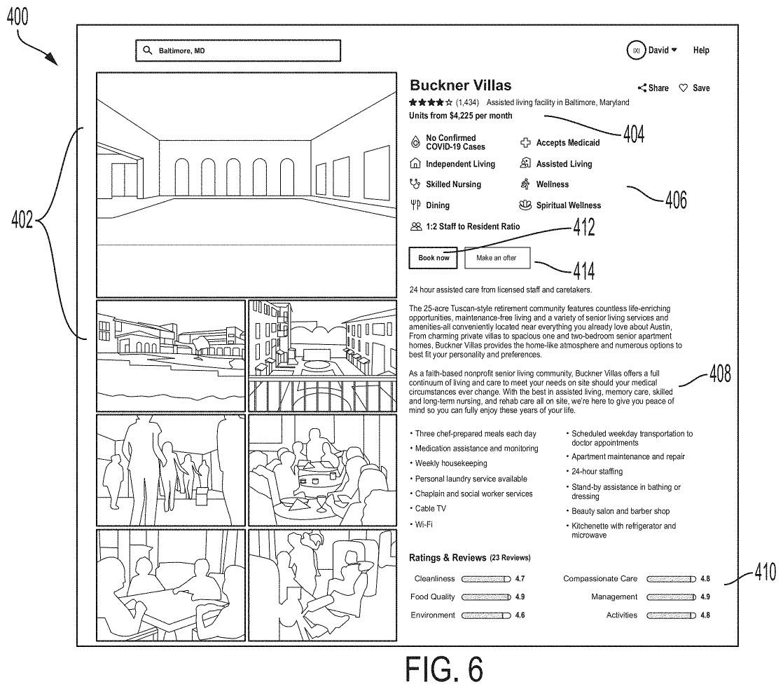

In some embodiments, upon selection of a provider facility from list 304, the first graphical user interface 120 may be configured to display an information page 400 for the selected provider facility. FIG. 6 shows an example of an information page for a selected provider facility, according to some embodiments. In some embodiments, only a registered user that is logged in can have access to an informational page 400. If a registered user is not logged in, the system may request the user to log in upon selection of a provider facility from list 304. The information page 400 may include one or more photos of the facility 402, a minimum rate for a bed or unit that is available at the facility 404, a brief description of amenities, services, offerings 406, a detailed description of amenities, services, offerings 408, resident reviews and ratings of the facility 410, a user-selectable booking icon 412, a user-selectable offer icon 414, and information regarding team members associated with the selected facility.

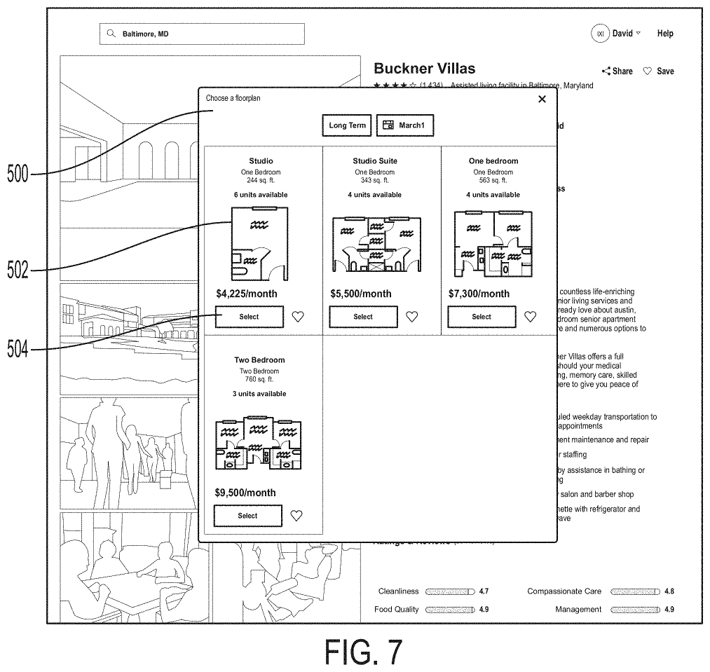

In some embodiments, upon user selection of the booking icon 412, the first graphical user interface 120 may be configured to display one or more pages for confirming booking selection. For example, the first graphical user interface may be configured to display a room selection page, a level of care selection page, and an additional services page. FIG. 7 shows an example of a room selection page 500, according to some embodiments. The information page 500 for the selected provider facility may include a selection of room type and floorplan 502. The room type may be, for example, a shared room, a studio, a one bedroom, a two bedroom, a three bedroom floor plan, etc. There may be one or more floor plans for a given room type. The room selection page 500 may include a user-selectable price 504 for each floor plan available.

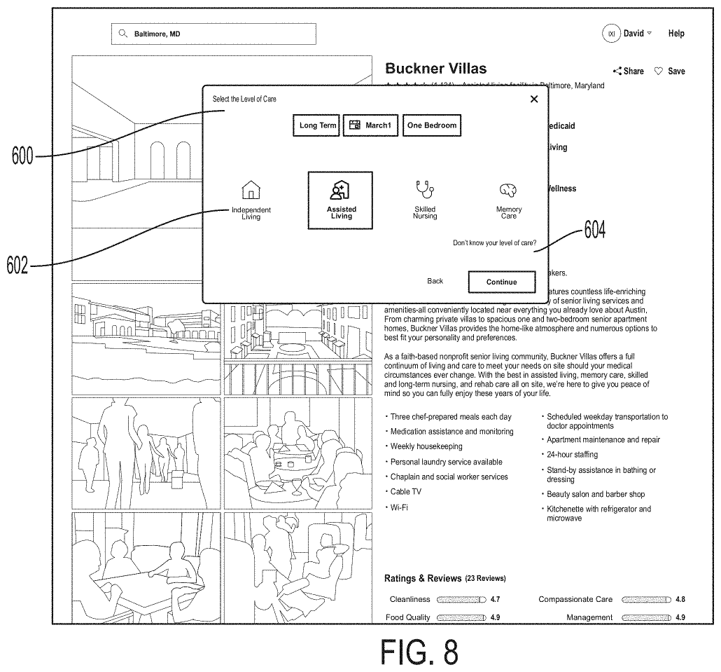

FIG. 8 shows an example of a level of care selection page 600, according to some embodiments. A level of care selection page 600 may include options to select an appropriate level of care for the person looking to become a resident at the selected facility. If the appropriate level of care is unknown, the user may select a recommendation icon 604. Upon user selection of the recommendation icon 604, the system may be configured to request responses from the user to assess a preliminary level of care to provide a recommendation for an appropriate level of care. In some embodiments, the recommendation may be generated prior to generating the resident application. In some embodiments, the recommendation may be part of the resident application.

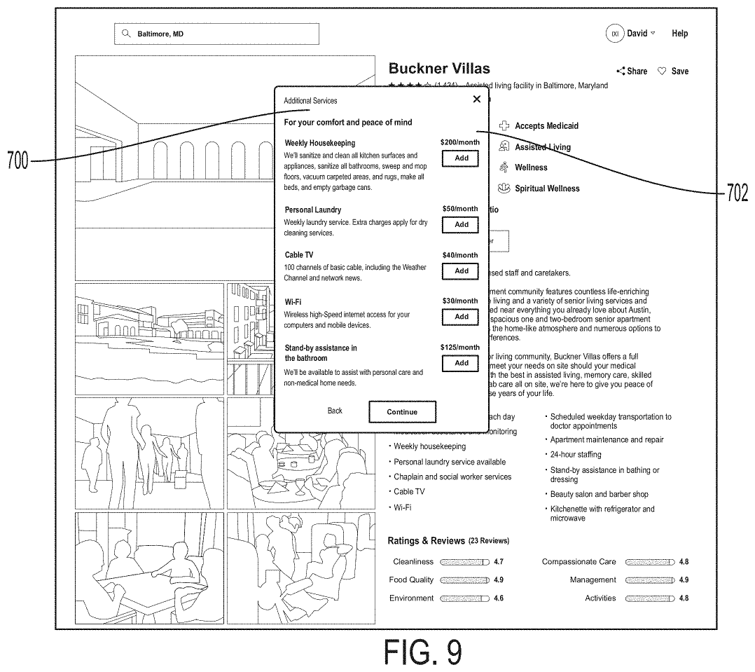

FIG. 9 shows an example of an additional services page 700, according to some embodiments. The additional services page 700 may include services or amenity options that may be added at an additional cost, such as weekly housekeeping, personal laundry, cable TV, WI-FI, and stand-by assistance in the bathroom.

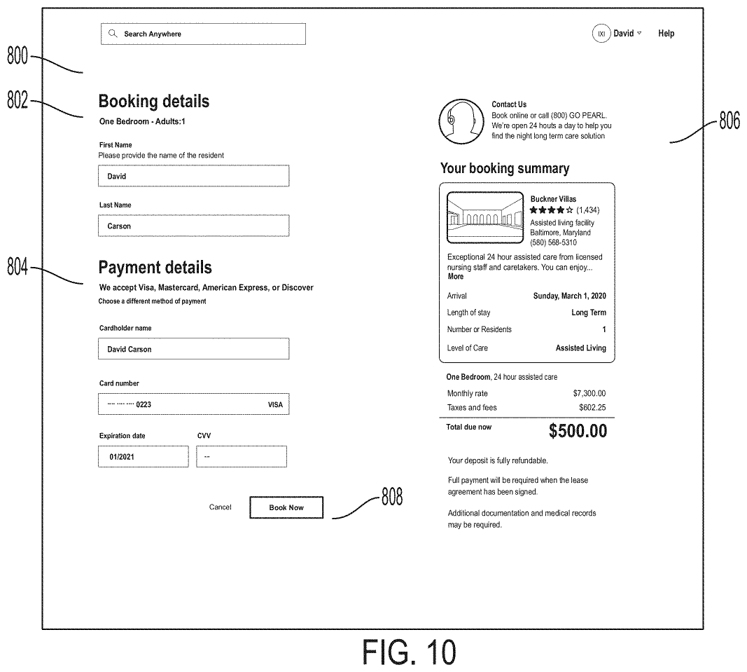

FIG. 10 shows an exemplary booking summary page 800, according to some embodiments. Upon user selection of booking preferences and options, for example via pages 400, 500, 600, and 700, a summary of the selection may appear on a booking summary page 800. The booking summary page 800 may prompt users to complete contact information 802 of the resident, payment information 804 to complete the booking, and a summary of selections and costs 806. The booking summary page 800 may include a book icon 808 that is configured, upon selection, to submit the booking selections to the selected facility for display on the second graphical user interface 140. The submitted booking may transmit application materials generated from the system's stored data on the prospective resident (“the common application”) for viewing on the second graphical user interface 140. The common application may include the information received from the user via the first graphical user interface 120. The information received from the user may include, for example, their demographic information and their healthcare & financial documents. In some embodiments, the information received from the user may be based at least in part on pages 400, 500, 600, and 700. The information received from the user may also include demographic data.

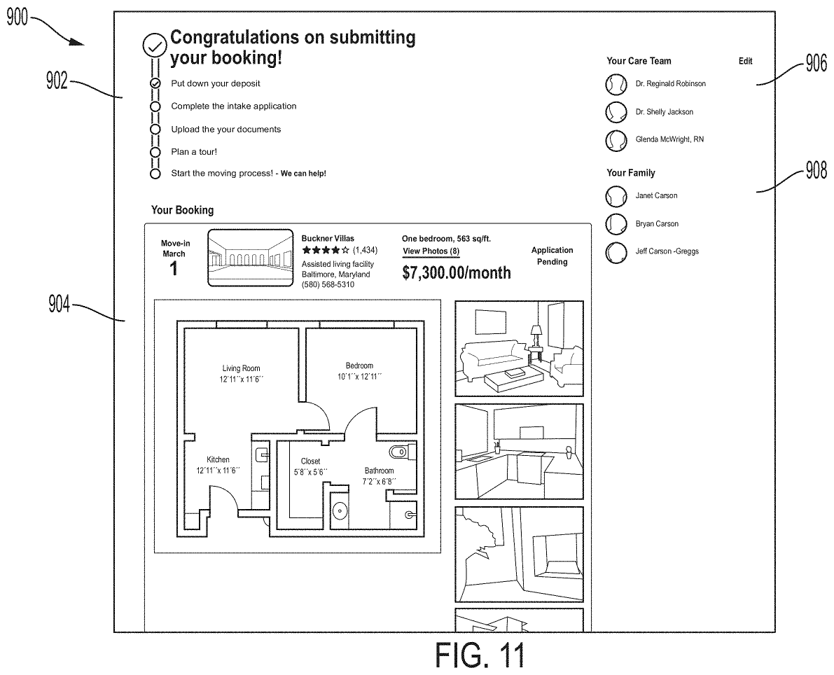

FIG. 11 shows an exemplary booking confirmation page 900 configured for display at the first graphical user interface for viewing by a consumer, according to some embodiments. In some embodiments, the booking confirmation may include a status of the booking and further steps the user will need to take in order to complete their booking 902, a summary of the booking 904, a list of care team members 906 for the resident at the booked facility, and a list of family members of the resident 908.

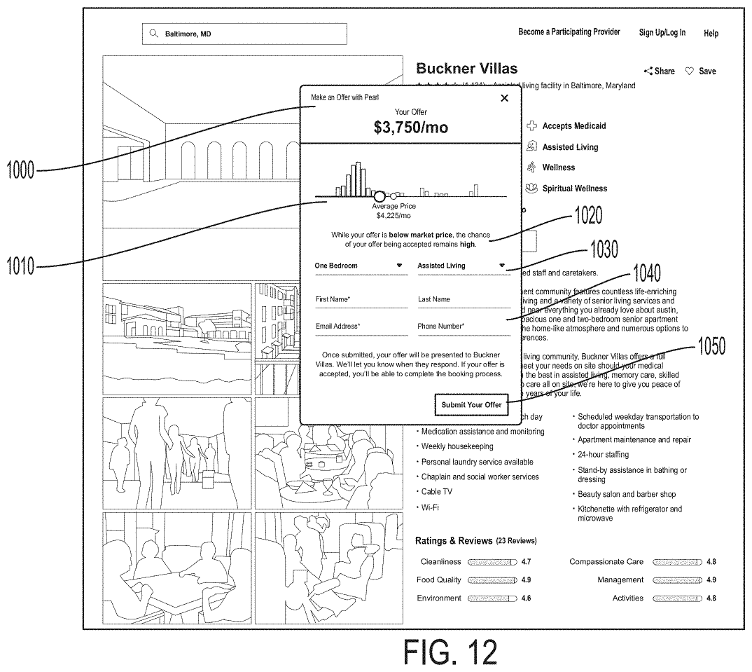

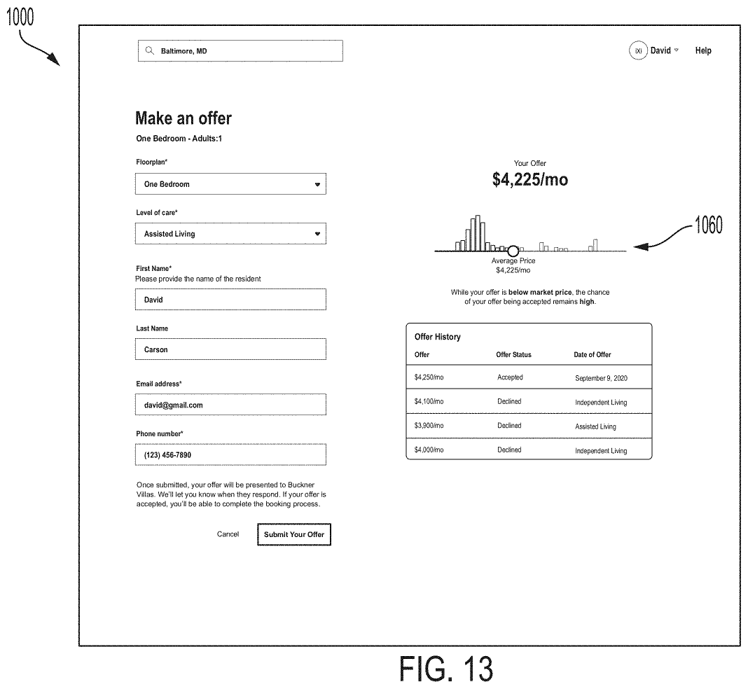

In some embodiments, upon user selection of a make an offer icon, such as offer icon 414, the first graphical user interface 120 may be configured to display an offer page 1000. FIG. 12 shows an exemplary make an offer page 1000, according to some embodiments. The offer page 1000 may include a diagram 1010 displaying the range of prior successful offer prices for a selected room and or unit. The diagram 1010 may include an indication of how many bids in a given price range are associated with the selected room and or unit or a room and or unit similar to the selected room and or unit. The offer page 1000 may include a brief statistical overview 1020 of a price offer received by the user. The brief statistical overview 1020 may describe whether the offer price is above market price, at market price, or below market price, and whether the chances of the offer being accepted is low or high. The offer page 1000 may include a brief description of the selection 1030, contact information 1040 for the user or resident, and a user-selectable submission icon 1050 configured to allow the user to submit their offer to the selected facility via the second graphical user interface 140. In some embodiments, in response to a user selection of the submission icon 1050, an application that includes information received from the user at the first graphical user interface 120 and the offer price may be automatically transmitted from the first graphical user interface to the second graphical user display for display on the second graphical user interface 140. The offer page 1000 may include an offer history 1060 for similar rooms at the selected facility. An example of an offer history 1060 is shown in FIG. 13, according to some embodiments. In some embodiments, the application may be received and displayed at the second graphical user interface 140. In response to a user of the second graphical user interface 140 selecting to accept the offer price and the application, the selected room may be booked for the consumer associated with the accepted application; pending consumer and provider approval. In some embodiments, the first graphical user interface 120 may be configured to receive and display one or more notifications from the second graphical user interface 140. One notification may include a message that the offer price submitted for a selected room has been accepted by the provider. In some embodiments, once the offer price has been accepted by a provider, the first graphical user interface 120 may be configured to display a page for completing the booking process. The page for completing the booking process may include a request for additional booking details that may include payment information.

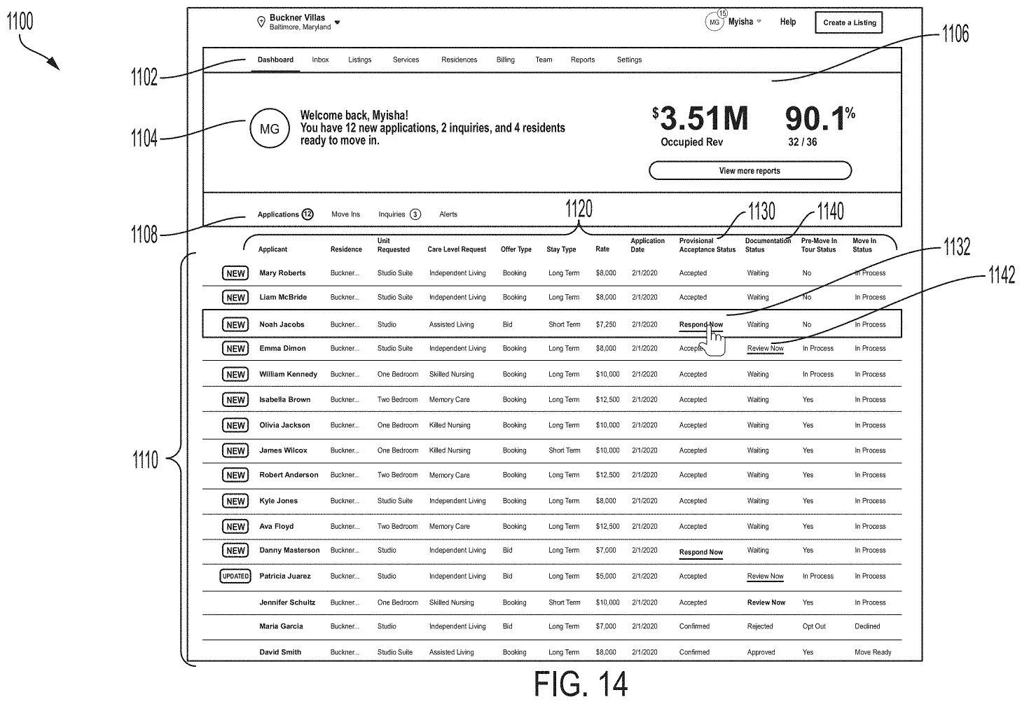

FIG. 14 shows an example of a landing page 1100 that may be displayed on the second graphical user interface 140, according to some embodiments. The landing page 1100 may be configured to display a dashboard 1102 that lists resident applications 1110 received from the first graphical user interface 120. The resident applications 1110 may be generated from user data on the platform, i.e. the common application. The dashboard 1100 may include an array of the received resident applications 1110 that includes a plurality of descriptors 1120 for each received resident application 1110. The plurality of descriptors 1120 may include, for example, an applicant name, residential facility, unit requested, care level requested, offer type (bid or booking), length of stay (long-term or short-term), price submitted, provisional acceptance status, documentation status, pre-move in tour status, and move-in status. The provisional acceptance status section 1130 may include a user-selectable icon 1132 for reviewing, accepting, responding, and updating the status of each application 1110. Upon selection of the user-selectable icon 1132 by a user representing the provider facility, the user may update a status of the consumer's application by indicating whether the application is accepted (refers to bids), confirmed (refers to booking), rejected, or a counter offer has been presented (refers to bids). The documentation status section 1140 may include a user-selectable icon 1142 for responding to documentation associated with each resident application 1110. Upon selection of the user-selectable icon 1142 by a user representing the provider facility, the user may update the status of the documentation review process by indicating whether the documentation has been received and approved and/or if the documentation is in process.

In some embodiments, the landing page 1100 may include a summary 1104 of new resident applications received, inquires, and number of residents ready to move-in. In some embodiments, the landing page 1100 may include economic information 1106 associated with occupancy of a provider facility. The economic information 1106 may include an occupancy rate and occupied revenue. In some embodiments, the landing page 1100 may include a selectable menu bar 1108, that when selected, may be configured to display resident applications 1110, inquiries from current or future residents, and move-in information associated with future residents. In some embodiments, upon user selection to accept an application via icon 1132 and accept documentation via 1142, then the system may be configured to allow one or more of the following to occur: a transaction record may be created, the listing of the room may be removed, a move-in record may be created, the application may be deleted or archived, confirmation of approval may be sent to a provider team, notification of successful approval and move-in may be sent to the consumer and the consumer's care team via the first graphical user interface 120, and a workflow management and reminders may be sent to the provider and consumer to ensure everything is move-in ready for the new resident.

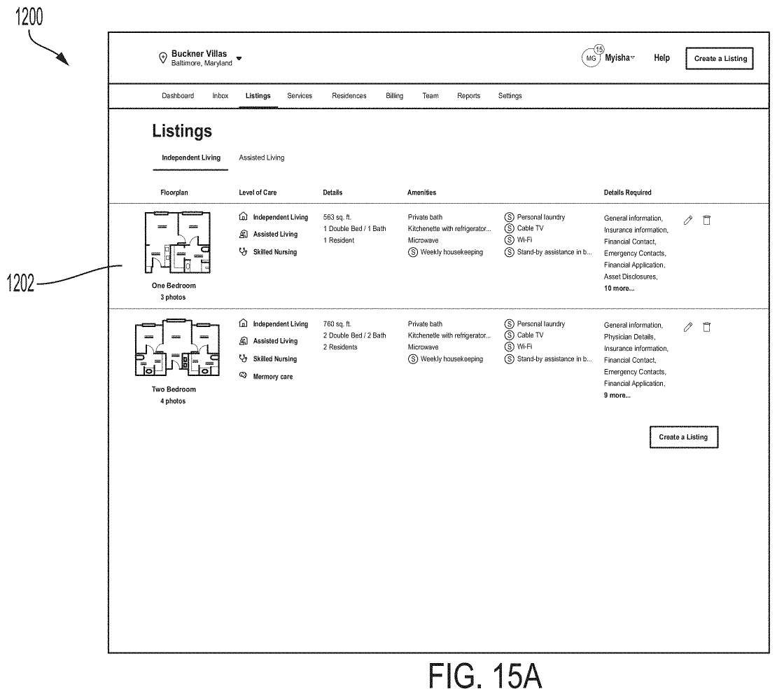

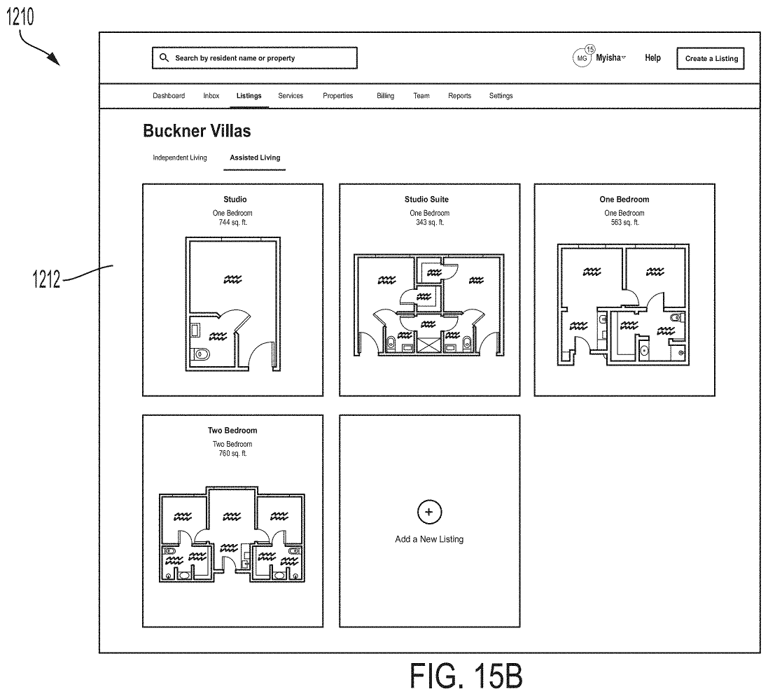

In some embodiments, the landing page 1100 of the second graphical user interface 140 may be configured to display listings and display a series of questions for creating a new listing. The landing page may display listings that are actively posted for a provider facility. FIG. 15A shows an example of a listings page 1200 of the second graphical user interface 140, according to some embodiments. The listings page 1200 may be configured to display unit listings that are posted or that can be posted and/or edited for a provider facility. The listing page 1200 may include a description 1202 of one or more of floor plans, levels of care available, room details (such as size and number of residents), amenities, and details required for a complete application. FIG. 15B shows another example of a listing page 1210 that includes a description 1212 of available listings via floor plan images.

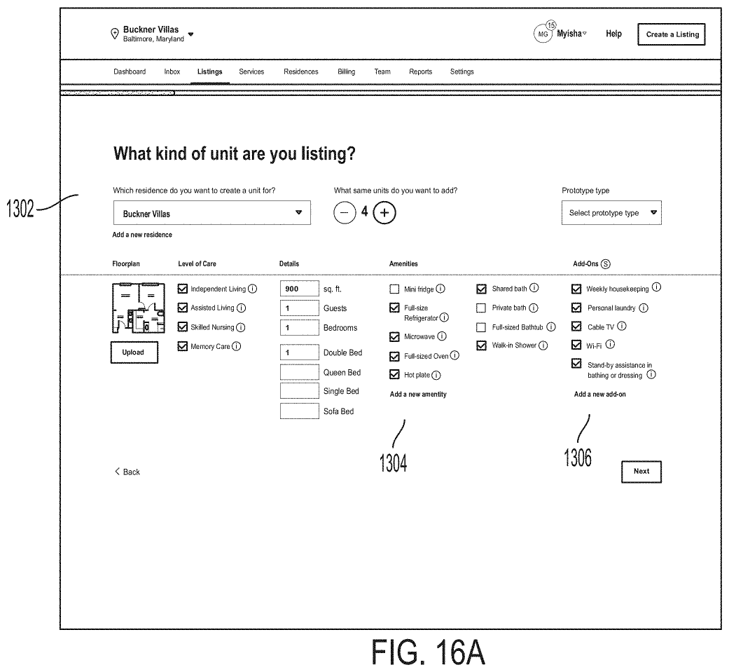

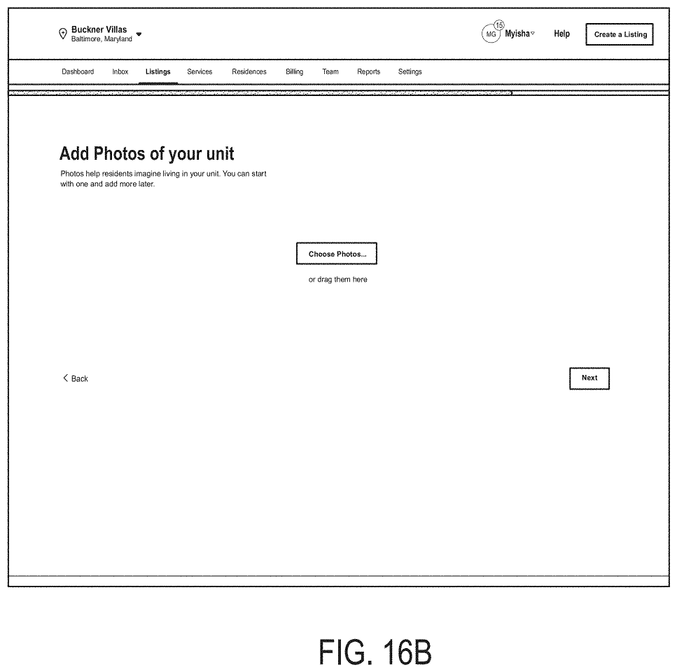

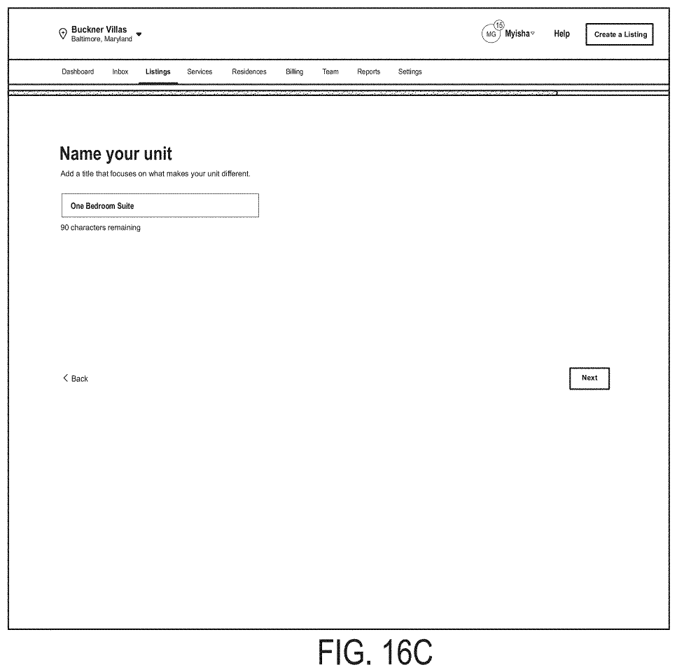

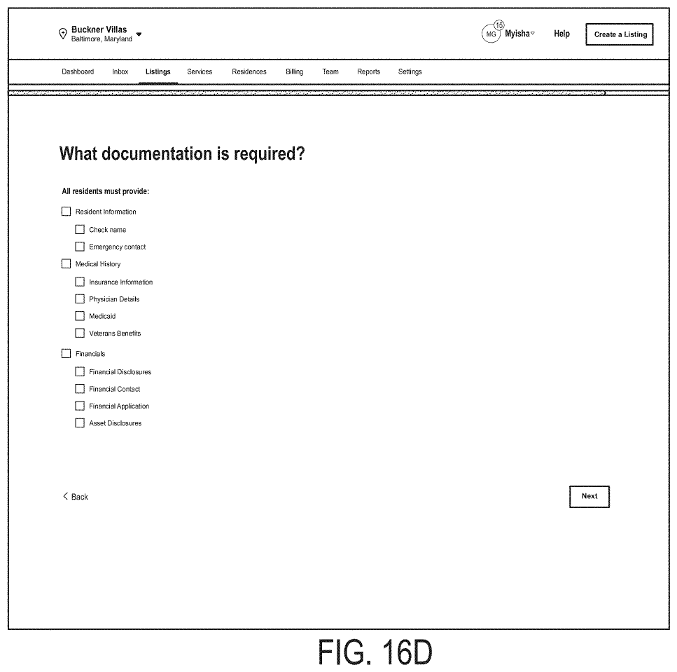

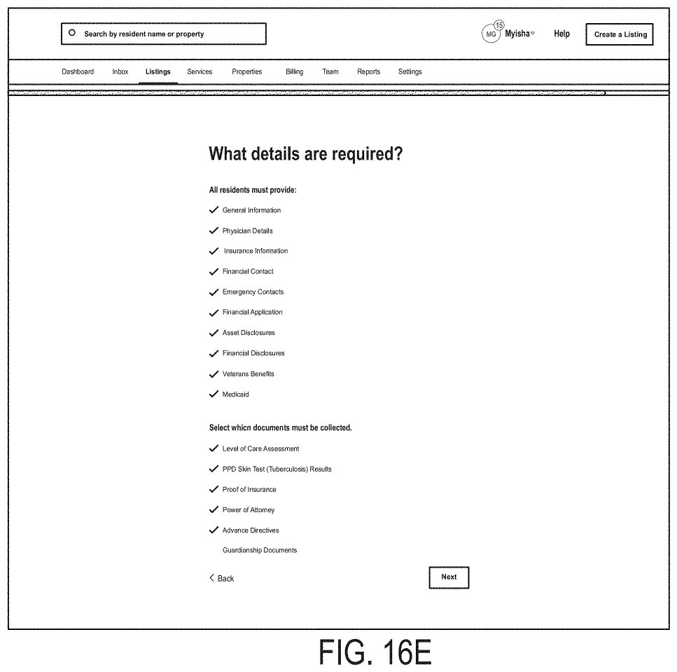

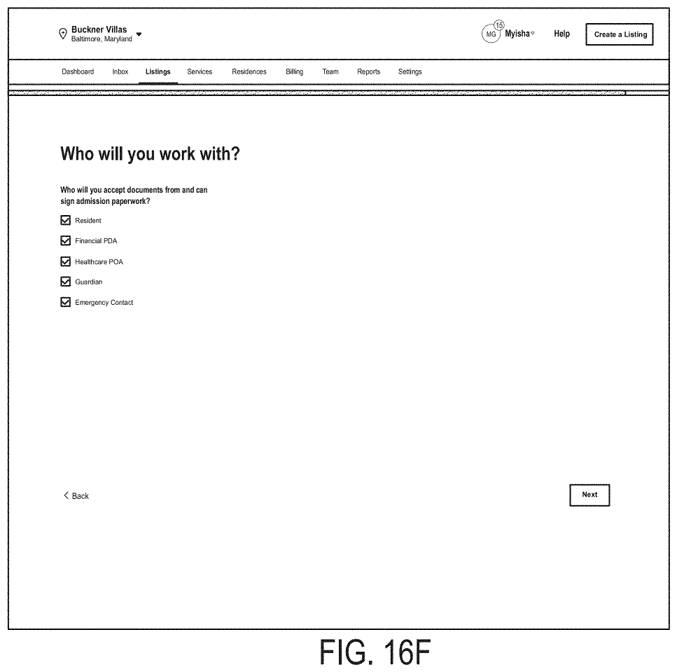

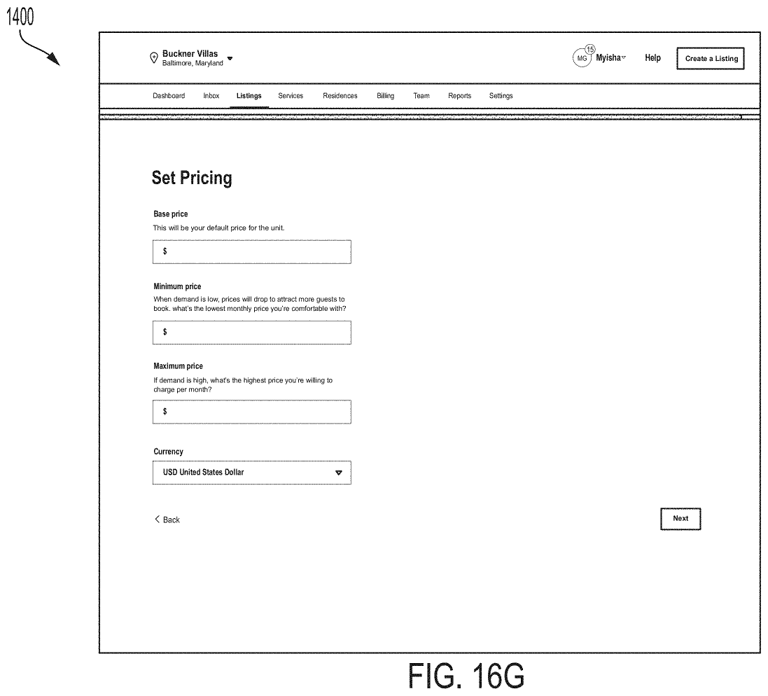

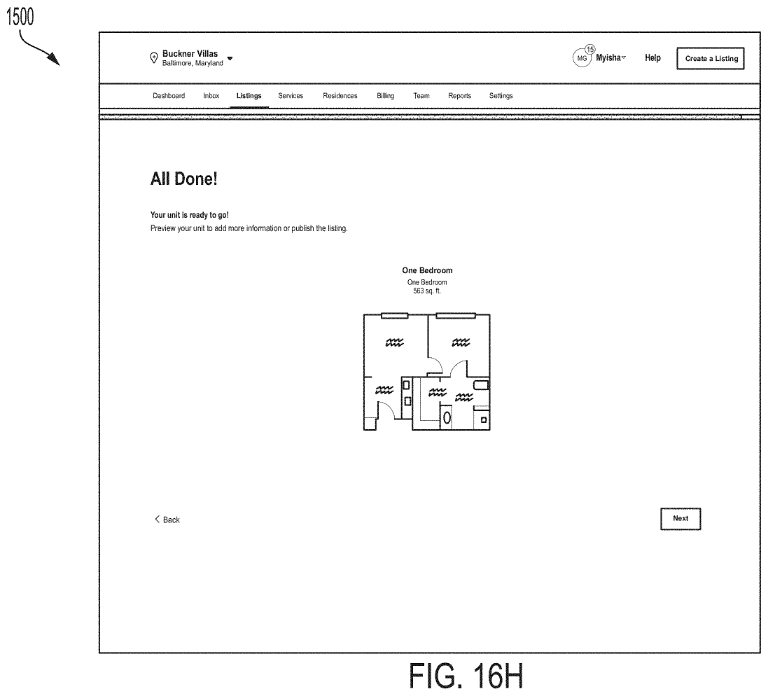

FIGS. 16A-16H show examples of questions or statements associated with listings page 1200, 1210 for creating a listing advertisement of an available room and or unit at a provider facility, according to some embodiments. FIG. 16A shows a “create a listing” page 1302 that includes user-selectable icons or user-fillable fields that may be respectively selected or filled by a user of a graphical user interface configured for a provider (such as a user of the graphical user interface 140) to populate information regarding the available room and or unit. For example, a floor plan or photo may be uploaded, a level or levels of care may be selected, room details (such as square footage, size, number of residents, number of bedroom, number and type of beds) may be provided, available amenities may be provided, and options for adding additional amenities for an added cost may be provided. Amenities may include, for example, a mini fridge, full-sized refrigerator, microwave, full-sized oven, hot plate, shared bath, private bath, full-sized bathtub, walk-in shower, air conditioning, heat, three chef-prepared meals each day, medication assistance and monitoring, weekly housekeeping, personal laundry services, chaplain and social worked services, cable TV, WI-FI, scheduled weekday transportation to doctor appointments, maintenance and repair, 24-hour staffing, stand-by assistance in bathing and dressing, beauty salon and barber shop, and kitchenette with refrigerator and microwave. The “create a listing” page 1302 may include a first option 1304 to add an amenity not listed, and a second option 1306 to add an additional amenity that may incur an additional cost.

FIG. 16B shows an example of an option to add a photo to a listing, according to some embodiments. FIG. 16C shows an example of an option to add a name to a listing, according to some embodiments. FIG. 16D shows an example of an option to select documentation that is required for a complete application, according to some embodiments. Required documentation may include, for example: resident information that may include resident name and emergency contact; medical history that may include insurance information, physical details, Medicaid, and veteran's benefits; financials that may include financial disclosures, financial contact, financial application, and asset disclosures. The page may include an option to add required documentation that is not listed. FIG. 16E shows an example of a summary of details required and documentation to be collected, according to some embodiments. Documentation to be collected may include, for example a level of care assessment, PPD skin test (Tuberculosis) results, proof of insurance, power of attorney, advance directives, and guardianship documents. FIG. 16F shows an example of who will be authorized to submit and/or view documents and sign admission documents on behalf of the resident, if not the resident themselves, according to some embodiments. Persons who will be authorized to submit documents, make decisions, and sign admission documents may include, for example, one or more of a resident, financial POA, healthcare POA, guardian, and/or an emergency contact.

FIG. 16G shows an example of a price setting page 1400 configured to receive from a user of the second graphical user interface 140 a base price, a minimum price, and a maximum price of an available unit that is to be listed. The base price may be a default price for the unit. The price of the unit may fluctuate based on demand between the minimum and maximum price. When demand is low, prices may drop to attract more guests to book. The minimum price is the lowest monthly price that the provider facility is willing to accept. When demand is high, prices may increase. The maximum price is the highest monthly price that the provider facility is willing to charge (for example when demand is high). In some embodiments, the base, minimum and maximum prices may be the same. In some embodiments, providers commit unoccupied units to the system via the second graphical user interface 140. Providers set floor bid prices they will accept for the units submitted via the second graphical user interface 140. In some embodiments, the floor bid price may be the minimum price. In some embodiments, a provider may choose whether to accept bids on a room or unit when listing the room or unit. In some embodiments, only price information submitted from consumers that meet the floor requirements set by the providers are displayed to consumers via the first graphical user interface 120. In some embodiments, only available rooms that satisfy price information set by a consumer using the first graphical user interface 120 may be displayed on the first graphical user interface 120 in response to a consumer search. In some embodiments, the price information may be a number or a number range. In some embodiments, price information set by a consumer may include a lower price point and a higher price point. In some embodiments, satisfying the price budget set by the consumer search includes that the lower price point of the price budget is at or above the floor bid or the minimum price set by a user of the second graphical user interface 140.

In some embodiments, upon completion of a listing process (for example, as outlined in FIGS. 15A-15B and 16A-16H), an advertisement for the listing may be generated. FIG. 16H shows a listing completion page 1500, according to some embodiments. The listing completion page 1500 may include that the unit listing may be published for customers to view via the first graphical user interface 120 or that more information may be added to the unit listing as necessary.

In some embodiments, in response to receiving a search criteria at the first graphical user interface 120 and in response to posting on a listing via the second graphical user interface 140 that satisfies the search criteria, the posted listing may be part of a list of available rooms displayable on the first graphical user interface 120. The search criteria may include a level of care and a price preference set by a user of the first graphical user interface 120. The price preference may include price information that is a range having a lower price point and a higher price point and may include an offer price. In some embodiments, in response to a user of the first graphical user interface 120 selecting the posted listing and selecting to submit a price preference for the posted listing, a resident bid offer may be automatically received at the second graphical user interface 140 and configured for display on the second graphical user interface 140. The resident application may include the price preference. In response to receiving the resident application and displaying the resident application at the second graphical user interface 140, the user of the second graphical user interface 140 may update the status of the application by indicating whether the application is accepted, confirmed, rejected or present a counter offer. In response to the status of the application being updated (for example via icon 1132 or icon 1142), a notification may be sent automatically for display on the first graphical user interface 120 for notifying the consumer. In some embodiments, the notification may be sent via email and/or text.

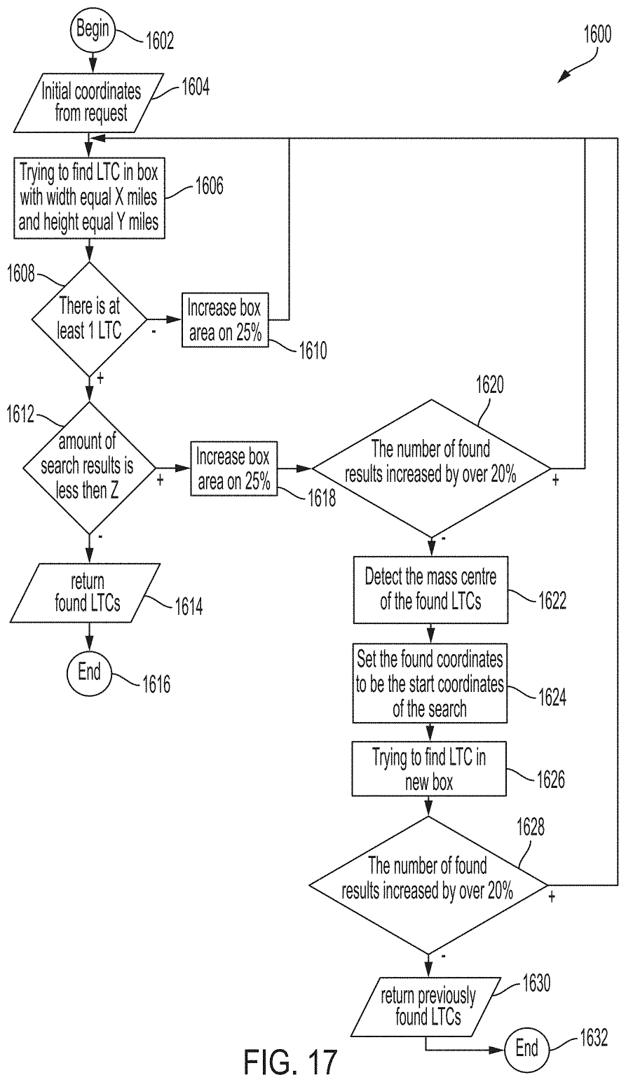

FIG. 17 shows an exemplary flowchart that describes a method 1600 for the automated creation and update of the map of search results for care facilities, according to some embodiments. Exemplary method 1600 may be implemented in systems such as system 100. The care facility may be, for example a long-term or short-term care facility. The method utilizes a plurality of variables: X—width of search map; Y—height of search map; Z—minimal normal amount of search results. In some embodiments, the method enables the consumer to zoom in and zoom out of the search results map and see the relevant, updated set of providers. In some embodiments, the methods automatically scales based on the applied filters and results that the consumer selects from exemplary results page 300.

In some embodiments, an area of a search map be based on X and Y. For example, the area of the search may be calculated by multiplying X and Y. In some embodiments, the method may begin upon initiation of a user at a graphical user interface 120 configured for a consumer (such as graphical user interface 120). In some embodiments, the method may be initiated by receiving search criteria at the graphical user interface 120. In some embodiments, the search criteria may include one or more of a preferred location, dates of stay, care services, and price information (may include a price number or a price range). In some embodiments, the method 1600 may yield search results of care providers that have one or more available rooms that satisfy the search criteria and that are located within a search area. In some embodiments, the system may be configured to receive searchable care providers provided by care providers registered with the system. As described in detail above and further below, the searchable care providers may be searched based on location, price information, a level of care, location, etc.

The search results may include an amount of care providers that may be compared to one or more thresholds for determining decision steps (such as exemplary decision steps 1608, 1612, 1620, and 1628). In some embodiments, the system may be configured to count a number of the search results (such as a number of care providers that satisfy search criteria and is located within a location area) and subsequent determination steps. In some embodiments, the system may be configured to count a number of the search results for optimizing search results. In some embodiments, updating or changing the coordinates for a search may be followed by identifying care providers that have one or more available rooms that satisfy the search criteria and are located in an area associated with the updated or changed coordinates.

At step 1602, the method may begin. At step 1604, initial coordinates are received from a request. The initial coordinates (such as X and Y) may be based on the received search criteria. In some embodiments, the initial coordinates (such as X and Y) and number of minimum results Z may be received from a search service. In some embodiments, the initial coordinates and number of minimum results Z may be determined by: 1) The geographic center of the object the consumer is looking for (for example, a city or district); 2) A consumer-selected point on the map; and 3) Center of a map frame displayed on the first graphical user interface 120.

At step 1606, a search to find a care facility in a first location area based on the initial coordinates may be conducted. In some embodiments, the initial coordinates may include a width equal to X miles and a height equal to Y miles. At step 1608, it may be determined whether there is at least one care facility within the first location area. If there is not at least one care facility within the first location area, at step 1610, the first location area may be increased by a first percentage to a second location area. The second location area may be associated with a second set of coordinates. At step 1606, if applicable, one or more of the second location area and the second set of coordinates associated with the second location area may be received. The second location area may have an area that is larger than the first location area by the first percentage of the first location area. In some embodiments, if at step 1606, one or more of the second location area and the second set of coordinates associated with the second location area is received, then the second location area may replace the first location area for the determination step of step 1608. In some embodiments, if at step 1606, one or more of the second location area and the second set of coordinates associated with the second location area is received, then the second set of coordinates associated with the second location area may replace the first set of coordinates for the determination step of step 1608. In some embodiments, the first percentage may be at least 10%, 15%, or 20%. In some embodiments, the first percentage may be at most 35%, 30%, or 25%. In some embodiments, the first percentage may be 10-35%, 15-30%, or 20-25%.

At step 1612, it may be determined whether an amount of search results is less than, greater than, or equal to Z. If it is determined that an amount of search results is greater than Z, then at step 1614, the found search results of care facilities may be returned for display to a user. In some embodiments, the found search results may be displayed automatically. At step 1616, the method may end. In some embodiments, the amount of search results associated with step 1612 may be an amount of first care providers. The amount of first care providers may be located in the first location area associated with the initial coordinates.

At step 1612, if it is determined that an amount of search results is less than Z, then at step 1618, the first location area may be increased by a second percentage to a third location area. The third location area may be associated with a third set of coordinates. In some embodiments, an amount of second care providers may be located in the third location area. In some embodiments, the second percentage may be at least 10%, 15%, or 20%. In some embodiments, the second percentage may be at most 35%, 30%, or 25%. In some embodiments, the second percentage may be 10-35%, 15-30%, or 20-25%

At step 1620, it may be determined whether or not a number of search results increased by over a third percentage as a result of increasing the first location area to the third location area. For example, it may be determined whether or not the amount of second care providers is an increased amount (increased by over the third percentage) of care providers compared to the amount of first care providers. In some embodiments, at step 1620, it may be determined whether or not a number of search results increased by over the third percentage as a result of increasing the first location area to the third location area. In some embodiments, the third percentage may be at least 5%, 10%, or 15%. In some embodiments, the third percentage may be at most 30%, 25%, or 20%. In some embodiments, the third percentage may be 5-30%, 10-25%, or 15-20%.

If it is determined at step 1620 that a number of search results increased by over the third percentage, then at step 1606, one or more of the third location area and the third set of coordinates associated with the third location area may be received. For example, if it is determined at step 1620 that the amount of second care providers is an increased amount (increased by over the third percentage) of care providers compared to the amount of first care providers, then at step 1606, one or more of the third location area and the third set of coordinates associated with the third location area may be received. In some embodiments, if at step 1606, one or more of the third location area and the third set of coordinates associated with the third location area, then the third location area may replace the first location area for the determination of step 1608. In some embodiments, if at step 1606, one or more of the third location area and the third set of coordinates associated with the third location area is received, then the third set of coordinates associated with the third location area may replace the first set of coordinates for the determination step of step 1608.

At step 1622, a center of a grouping of the search results (care providers) may be detected if it is determined at step 1620 that the number of search results did not increase by over a third percentage as a result of increasing the first location area to the third location area. That is, for example, that the amount of second care providers is not an increased amount (increased by over the third percentage) of care providers compared to the first amount of care providers. The center of the grouping of the search results may have center coordinates. At step 1624, the center coordinates may be set as a center for a fourth location area. The fourth location area may be associated with a fourth set of coordinates. In some embodiments, setting the center coordinates as the center for the fourth location area may cause a shift in a search range from a search range associated with the third location area to a search range associated with the fourth location area. In some embodiments, the fourth location area may have a geometrical area that is equal to a geometrical area of the third location area.

At step 1626, a search to find a care facility in the fourth location area may be conducted. At step 1628, it may be determined whether or not a number of search results in the fourth location area increased by over a fourth percentage compared to a number of search results associated with the third location area. The number of search results in the fourth location area may be an amount of third care providers. In some embodiments, it may be determined at step 1628 whether or not the amount of third care providers is an increased amount (increased by over the fourth percentage) of care providers compared to the amount of second care providers.

If it is determined that the number of search results in the fourth location area is not an increase by over the fourth percentage compared to the number of search results in the third location area, then at step 1630, previously found search results (care facilities that satisfy search criteria and are located in the third location area) may be returned for display. For example, if it is determined that the amount of third care providers in the fourth location area is not an increased amount (by over the fourth percentage) in a number of care providers compared to the amount of second care providers in the third location area, then at step 1630, previously found search results (care facilities that satisfy search criteria and are located in the third location area) may be returned for display. In some embodiments, the previously found search results may be automatically displayed on the first graphical user interface configured for a consumer. In some embodiments, the displayed search results as a part of a user-selectable list of care providers. In some embodiments, the search results may be displayed automatically as a part of a user-selectable list of care providers.

If it is determined that the amount of third care providers in the fourth location area is an increased number (by over the fourth percentage) of care providers compared to the amount of second care providers in the third location area, then at step 1606, one or more of the fourth location area and the fourth set of coordinates may be received. For example, if it is determined that the amount of third care providers in the fourth location area is an increased number (by over the fourth percentage) of care providers compared to the amount of second care providers in the third location area, then at step 1606, one or more of the fourth location area and the fourth set of coordinates may be received. In some embodiments, if at step 1606, one or more of the fourth location area and the fourth set of coordinates, then the fourth location area may replace the first location area for the determination step of step 1608. In some embodiments, if at step 1606, one or more of the fourth location area and the fourth set of coordinates, then the fourth set of coordinates may replace the first set of coordinates for the determination step of step 1608. In some embodiments, the fourth percentage may be at least 5%, 10%, or 15%. In some embodiments, the fourth percentage may be at most 30%, 25%, or 20%. In some embodiments, the fourth percentage may be 5-30%, 10-25%, or 15-20%. At step 1632, the method may end.

In some embodiments, an architecture of the system (such as system 100) may be modeled from and leverage a cloud-native template and or solution. For example, the architecture of the system may leverage and instantiate AWS' HIPAA Reference Architecture. The system may be built using a plurality of frontend and backend technologies. One exemplar embodiment may utilize Python, mySQL/MongoDB, NodeJS, HTML5/CSS/JS. In some embodiments, the system has the following components: a system of relational and noSQL databases; a data API for inventory management; an API for consumer requests; a mail server to facilitate messages between all users of the system; a calendar service for coordination of offline application services for consumers; a search engine user interface—for consumers; a bidding system for care—for consumers; an inventory management user interface—for providers; and a business optimization system—for providers.

In some embodiments, the platform may include a secure storage system (such as ZeroDark Cloud and secured Amazon S3), an online payment processing service (such as www.escrow.com, stripe.com, authorize.net, payoneer.com, 2checkout.com and square.com), a secure authentication service (such as private.id secure biometric authentication service), and a standards-based data interoperability layer (such as HL7 FHIR).

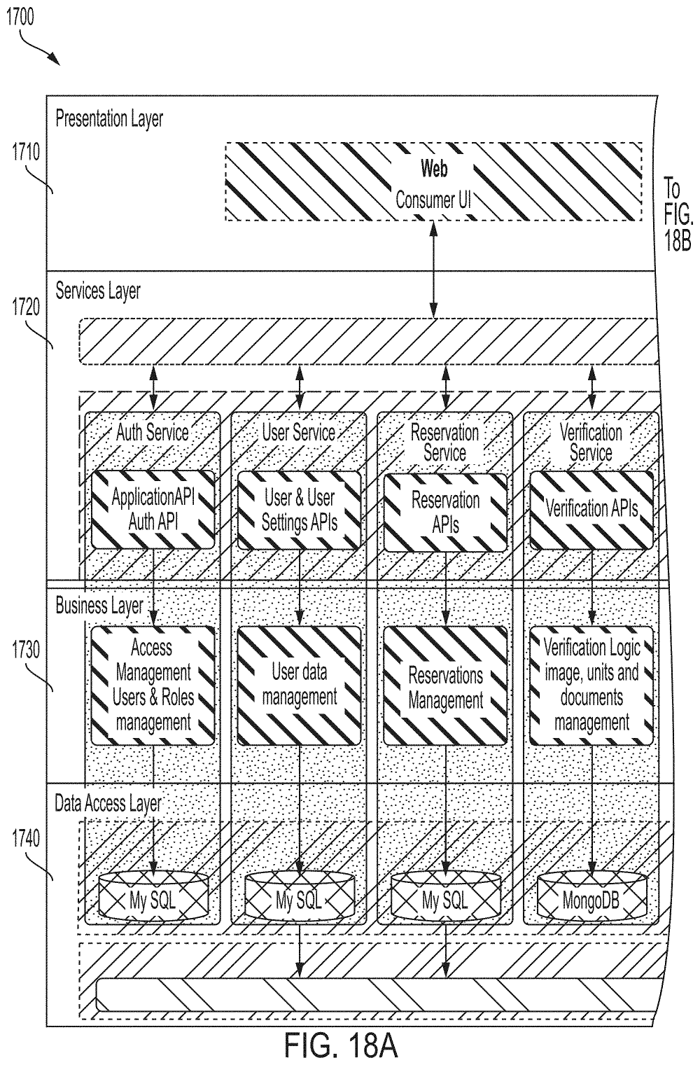

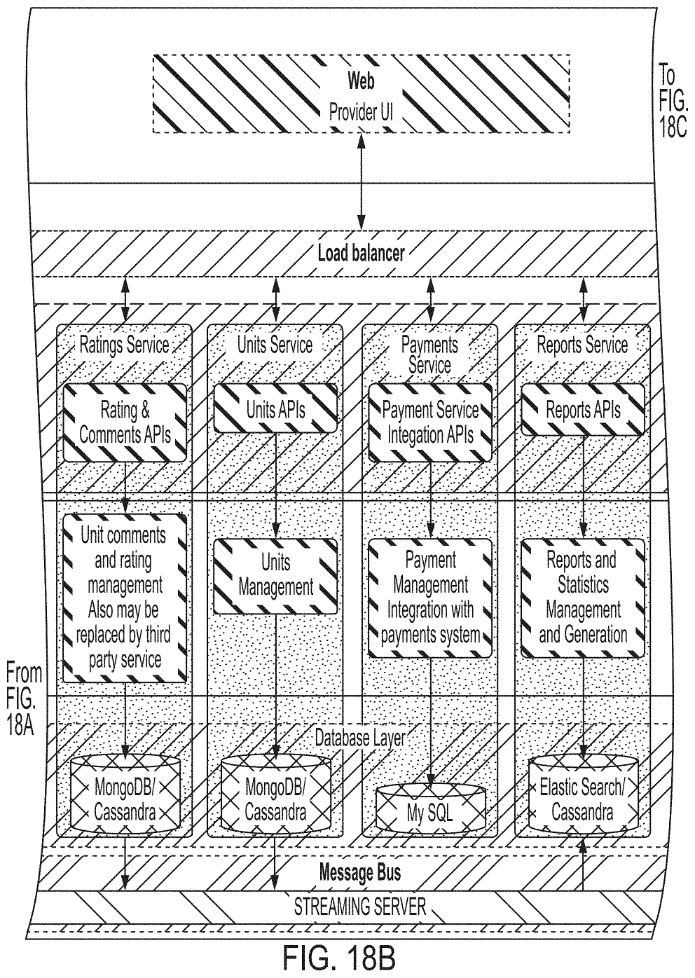

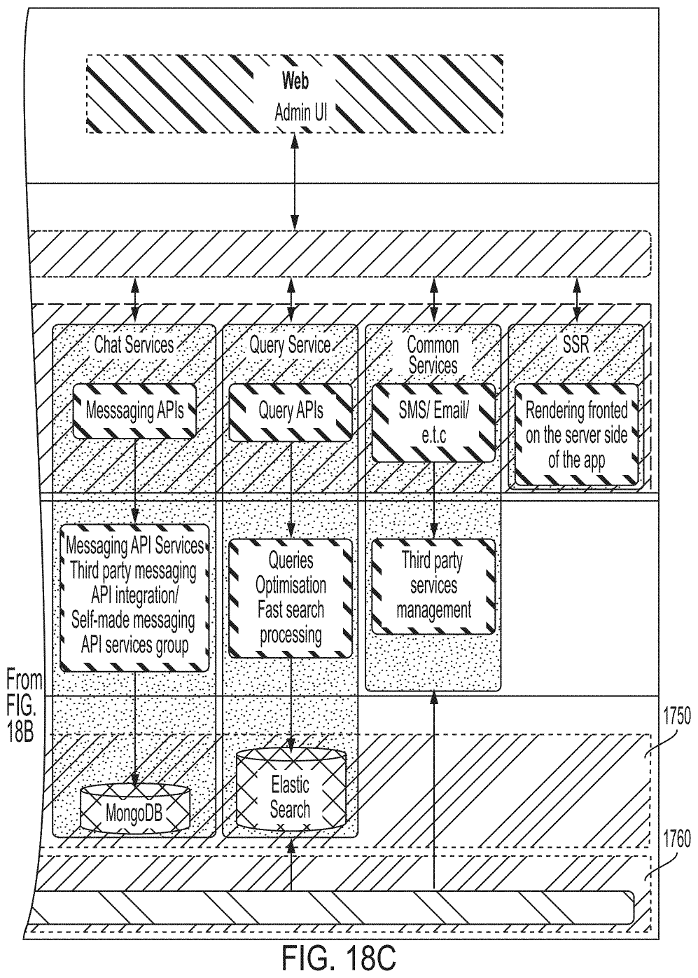

FIGS. 18A-18C show an exemplary flowchart of a high level architecture 1700 of a communications flow of a system (such as system 100), according to some embodiments. In some embodiments, the high level architecture may include a presentation layer 1710, a services layer 1720, a business layer 1730, and a data access layer 1740. The data access layer may include a database layer 1750 and a message bus 1760.

In some embodiments, requests to an application program interface associated with the system (such as system 100), the business layer 1730, and the data access layer 1740 may be executed asynchronously. The communication between services may be organized using the message bus 1740 using RPC (Remote procedure calls) protocols, such as gRPC. In some embodiments, all communication services may be implemented using the following layers: Presentation Layer configured to leverage design templates that generate the graphical user interface for the system. The Presentation layer may also include controllers that react to consumer actions and execute pre-defined interaction pathways. The Service Layer that abstracts low-level technical details and provides a uniformed interface to the system; The Business Layer contains the industry and operational logic and rules of the system. It may include methods and interfaces to be used by the Service Layer and coordinate data between Service Layer and Data Access Layer. The Business layer may include the following functionalities: users and roles management, units management, reservations management, report generation logic, chat functionality support, server-side rendering of the web UIs, query optimization logic, verification mechanism, unit rating and comments management, payment service integration, third party services (Email sender, SMS sender, etc.), and user data management. The Data Access Layer encapsulates all the data objects and stores the data needed for the operation of the system. An exemplary embodiment may be configured for TypeORM and Mongoose libraries to provide interaction with the database and object-relational mapping. The data access layer may prevent the direct access to the database from communication services; Database Layer may include multiple types of databases: MySQL or a similar relational database—for the most sensitive data and MongoDB/Cassandra or a similar NoSQL database—for less sensitive and most actively requested data; Elasticsearch—for not sensitive data, which requires very fast access. In some embodiments, the Data Access Layer may be the only layer that has access to the databases.

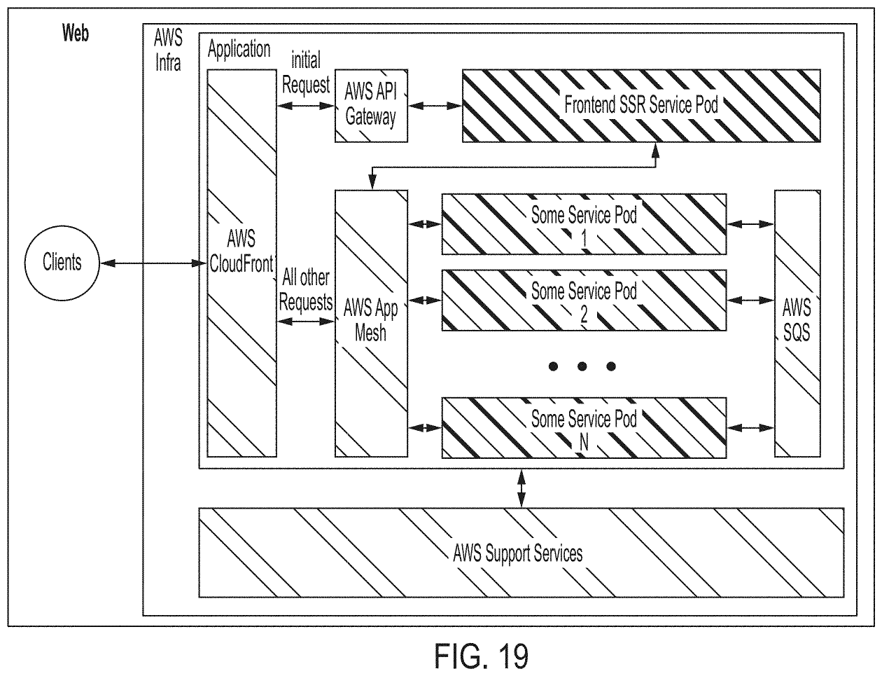

In some embodiments, a cloud platform solution may be used to implement the system (such as system 100). In some embodiments, the cloud platform solution may be Amazon Web Services (AWS). Other embodiments may leverage components from Google Cloud Platform, Microsoft Azure, or cloud providers. FIG. 19 shows an exemplar embodiment using AWS products and services. In some embodiments, the technical infrastructure includes: AWS API Gateway—the endpoint responsible for serving the frontend; AWS App Mesh—the service application that performs balancing, monitoring, tracing and logging of incoming requests. Also responsible for requests retry policy; Frontend SSR Service Pod—a special pod that is responsible for serving the frontend (the main difference of this pod is that it works directly with API Gateway and does not have or need a database cluster, but still has efficient caching via a Redis cluster); Some Service Pod—this is the usual pod of our application, the structure of such pods is described in more detail below; AWS SQS—Amazon data bus; AWS CloudFront—Used to ensure fast delivery of files to the client, to optimize API response times, and to gracefully mitigate the impact of denial of service attacks; AWS Support Services these are all the auxiliary services that AWS provides, a more detailed description of these services will be below.

In some embodiments, the cloud platform solution may be used for monitoring and tracking cloud services, text messaging, and storing files. For example, AWS CloudWatch may be used for monitoring and tracking, AWS SNS may be used for text messaging, and AWS S3 may be used for storing files.

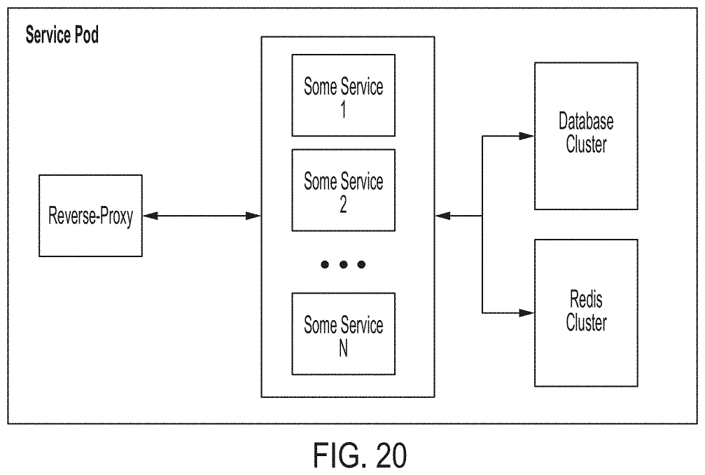

In some embodiments, a service pod (units computing) structure may be created and managed. FIG. 20 shows an example of a service pod, according to some embodiments. The service pod may include: Reverse-Proxy—pod element that is responsible for load balancing, retry policy, tracing and consistent logging of all incoming requests. A restriction may be imposed on this element in the form of the factor that the proxy must support work with the RPC protocol. In some embodiments, a proxy and communication bus (such as Envoy) may be used as a proxy because AWS uses it for the service mesh and it has excellent RPC support; Some Service—this is one or more services that implements business logic for running and maintaining the system (such as system 100); Database Cluster—the cluster of the database used by this service (which database will be used by the pod depends on which service it is); and Redis Cluster—a Redis database cluster used for caching recently retrieved data.

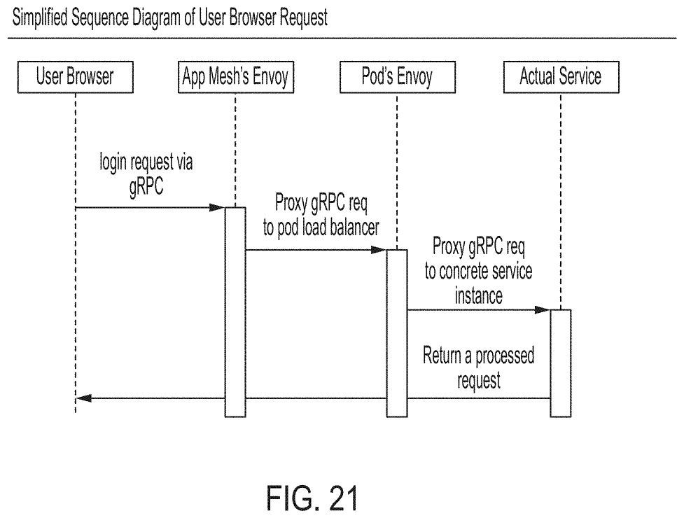

FIG. 21 shows an example of a sequence diagram of a user browser request, according to some embodiments. FIG. 21 illustrates a path a user request takes from a browser to a final service. In some embodiments, to ensure correct operation between the browser and the server, the user request may be sent through a proxy server (such as Envoy). In some embodiments, at least two proxy servers may be used so that a first server may be a single entry point for the entire API, and the second server may directly balance the load between the services of a particular pod, as well as retry policy. In some embodiments, user browser requests may be easy to monitor, trace, and log.

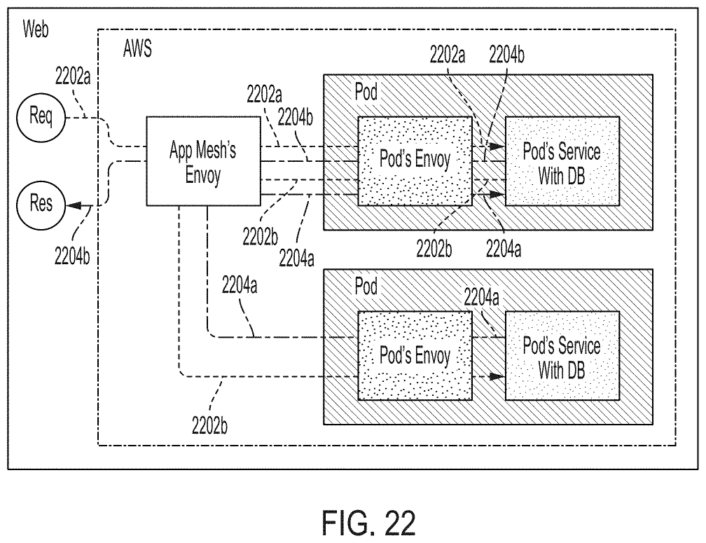

FIG. 22 shows an example of a path of a request, according to some embodiments. FIG. 22 illustrates the path of a request with a first line 2202a-b and a path of the response with a second line 2204a-b. In the example of FIG. 22, App Mesh's Envoy resolves the path to the required pod, and the pod's Envoy resolves and redirects to the appropriate service.

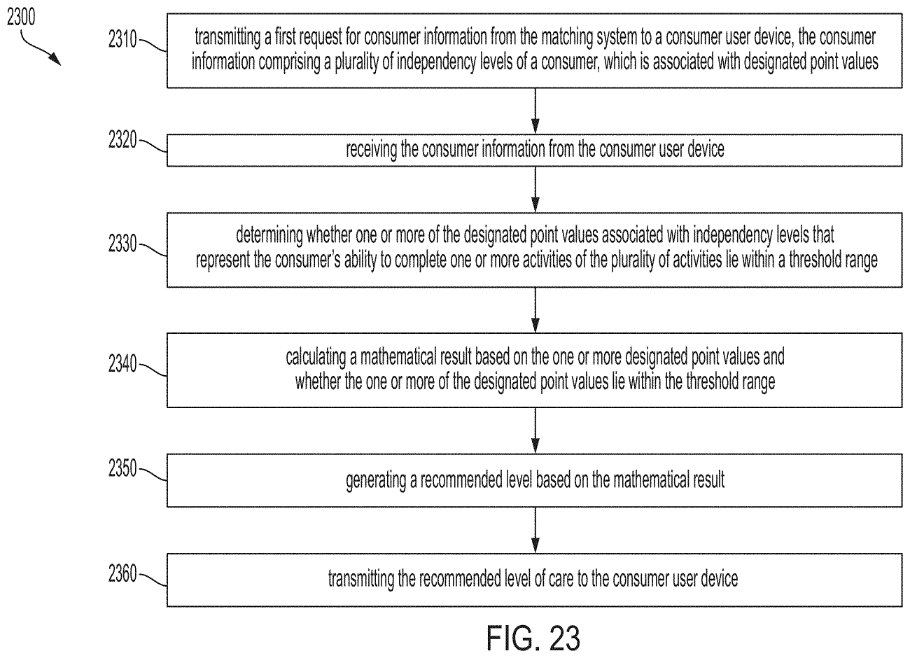

FIG. 23 shows a flowchart of an exemplary method 2300 for recommending a level of care for consumers, according to some embodiments. In some embodiments, the exemplary method 2300 may be performed on systems such as system 100. In some embodiments, in response to a consumer's selection of a recommendation icon (such as recommendation icon 208), the system may be configured to determine and provide a recommendation of a level of care based on a consumer's response to a plurality of statements or questions via exemplary method 2300. The recommendation for the level of care may be a preliminary level of care assessment (as described above in reference to recommendation icon 208) based on functional assessment data collected from the consumer via a graphical user interface. Details of the preliminary level of care assessment is provided above in reference to recommendation icon 208. For brevity, the function assessment areas and consumer response types associated with the preliminary level of care assessment will not be repeated since it was described above in reference to recommendation icon 208.

In some embodiments, the exemplary method shown in FIG. 23 may be performed using a computer system comprising one or more processor, memory, and a display, according to some embodiments. At step 2310, a first request for consumer information from the matching system to a consumer user device may be transmitted, the consumer information comprising a plurality of independency levels of a consumer, each independency level of the plurality of independency levels represents the consumer's ability to complete an activity of a plurality of activities, each independency level is associated with a designated point value. In some embodiments, the independency levels may be indicated through specific consumer responses. Each type of consumer response may be associated with a specific independency level of the future resident's ability to complete a particular activity. For example, a first response may be “always,” a second response may be “usually independent,” a third response may be “usually not,” and a fourth response may be “never.” Each response type may be associated with a designated score. For example “always” may be designated a score of 0, “usually independent” may be designated a score of 1, “usually not” may be designated a score of 2, and “never” may be designated a score of 3. According to some embodiments, the plurality of activities may be associated with functional assessment data of a preliminary level of care assessment (as discussed above in reference to icon 208). According to some embodiments, the one or more activities may be associated with memory care. In some embodiments, the one or more activities may be associated with non-memory care. According to some embodiments, the consumer user device may be configured to display the first request via a graphical user interface (such as interface 120).

At step 2320, the consumer information from the consumer user device may be received. At step 2330, a determination of whether one or more of the designated point values associated with independency levels that represent the consumer's ability to complete one or more activities of the plurality of activities lie within a threshold range may be made.

At step 2340, a mathematical result based on the one or more designated point values and whether the one or more of the designated point values lie within the threshold range may be calculated. According to some embodiments, the mathematical result may include an average of the designated scores. According to some embodiments, the mathematical result may be based on all the accumulated points and their evaluation against a continuum of thresholds. At step 2350, generating a recommended level based on the mathematical result. At step 2360, transmitting the recommended level of care to the consumer user device. According to some embodiments, the recommended level of care may be based on a plurality of care options that include one or more of memory care, independent living, assisted living, and skilled nursing.

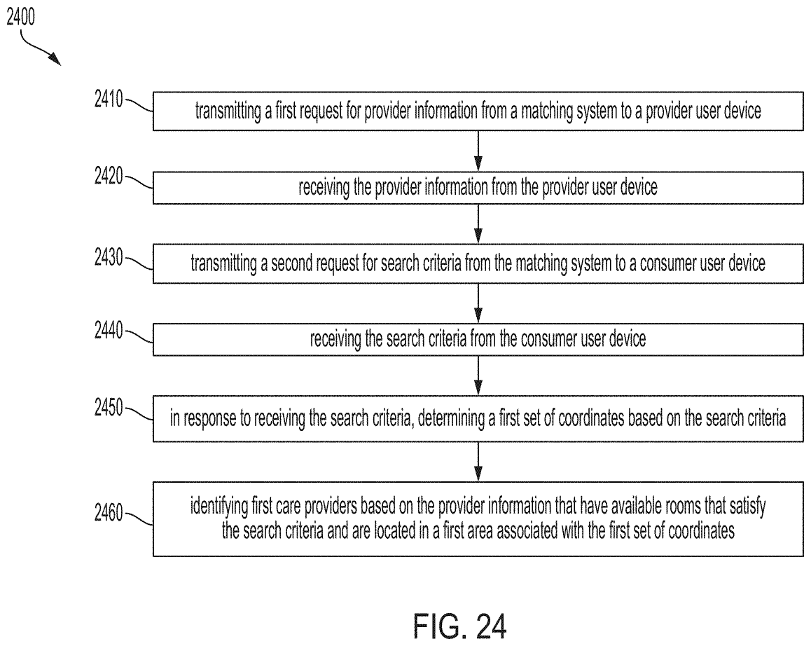

FIG. 24 shows a flowchart of an exemplary method 2400 for matching consumers with long term and short term care providers using a computer system comprising one or more processor, memory, and a display, according to some embodiments. Exemplary method 2400 may be implemented on systems such as system 100. According to some embodiments, exemplary method 2400 may incorporate one or more steps of method 1600 of FIG. 17. At step 2410, a first request for provider information from a matching system to a provider user device may be transmitted. According to some embodiments, the provider information may include information such as facilities, viewing pricing, services, photos, and reviews associated with the care facilities. According to some embodiments, the provider information may include information shown, for example, in FIGS. 15A-16H. According to some embodiments the provider user interface may be configured to display a graphical user interface (such as interface 140). At step 2420, the provider information from the provider user device may be received.

At step 2430, a second request for search criteria from the matching system to a consumer user device may be transmitted, the search criteria comprising at least one or more of a preferred location, price information, and consumer care needs. At step 2440, search criteria from the consumer user device may be received. According to some embodiments, search criteria may include, for example, information of the initial search information 206.

At step 2450, in response to receiving the search criteria, a first set of coordinates based on the search criteria may be determined. At step 2460, first care providers based on the provider information that have available rooms that satisfy the search criteria and are located in a first area associated with the first set of coordinates may be identified. In some embodiments, the first care providers is a candidate set that may be sent to the consumer user device for display textually and visually via a map (such as map 306) on a graphical user interface (such as interface 120) of the consumer user device.

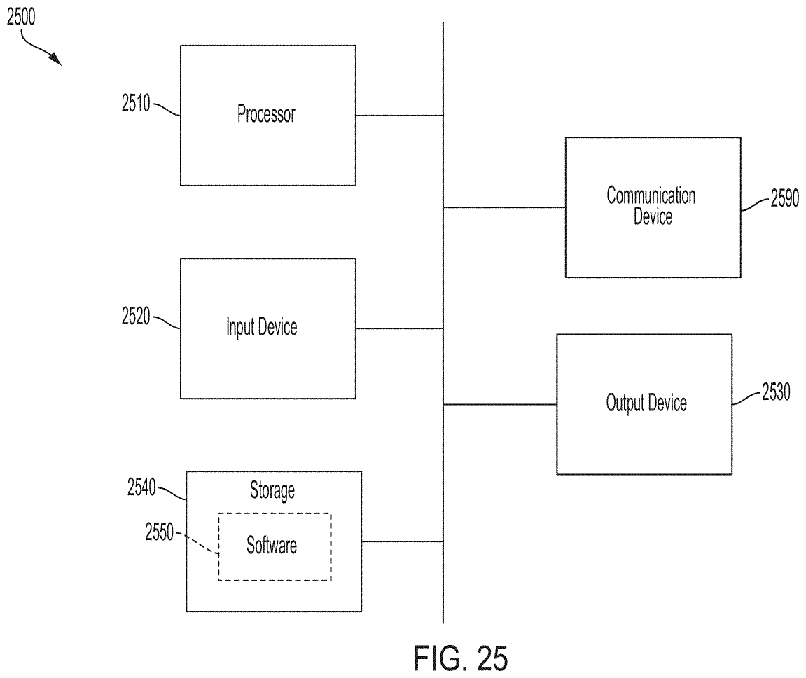

FIG. 25 illustrates an example of a computing device 2500 in accordance with some embodiments (such as system 100), or a computing device for implementing methods 2300 and 2400. Device 2500 can be a host computer connected to a network. Device 2500 can be a client computer or a server. As shown in FIG. 25, device 2500 can be any suitable type of microprocessor-based device, such as a personal computer, workstation, server, or handheld computing device (portable electronic device) such as a phone or tablet. The device can include, for example, one or more of processor 2510, input device 2520, output device 2530, storage 2540, and communication device 2560. Input device 2520 and output device 2530 can generally correspond to those described above and can either be connectable or integrated with the computer.

Input device 2520 can be any suitable device that provides input, such as a touch screen, keyboard or keypad, mouse, or voice-recognition device. Output device 2530 can be any suitable device that provides output, such as a touch screen, haptics device, or speaker.

Storage 2540 can be any suitable device that provides storage, such as an electrical, magnetic, or optical memory including a RAM, cache, hard drive, or removable storage disk. Communication device 2560 can include any suitable device capable of transmitting and receiving signals over a network, such as a network interface chip or device. The components of the computer can be connected in any suitable manner, such as via a physical bus or wirelessly.

Software 2550, which can be stored in storage 2540 and executed by processor 2510, can include, for example, the programming that embodies the functionality of the present disclosure (e.g., as embodied in the devices as described above).

Software 2550 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 2540, that can contain or store programming for use by or in connection with an instruction execution system, apparatus, or device.

Software 2550 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic, or infrared wired or wireless propagation medium.

Device 2500 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 2500 can implement any operating system suitable for operating on the network. Software 2550 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

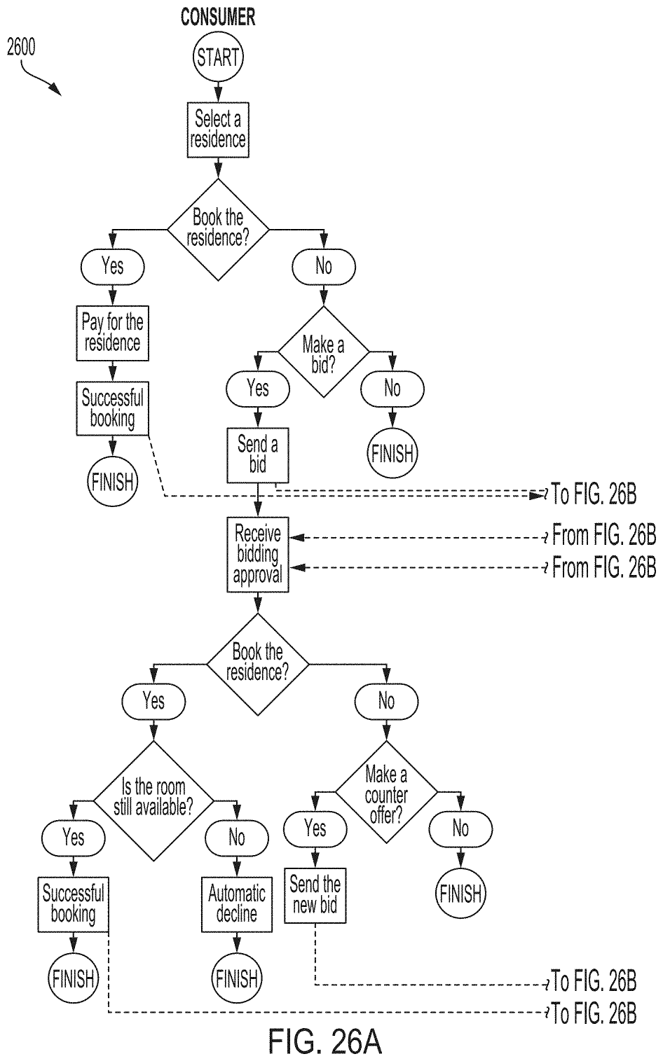

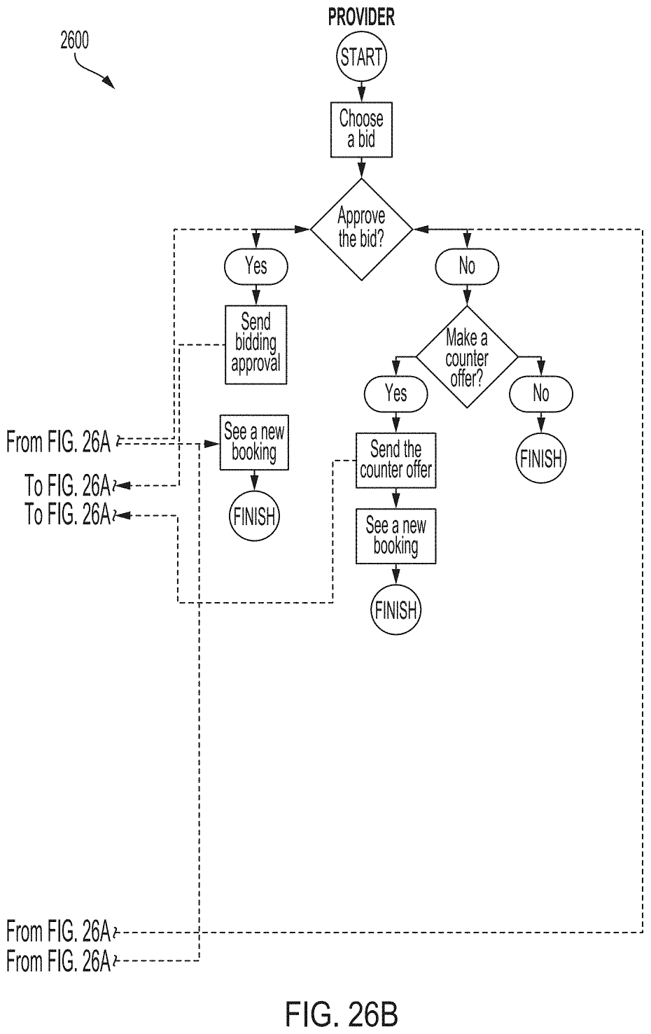

FIGS. 26A-26B show a flowchart that describes an example method 2600 for consumers finding, bidding and booking care providers, according to some embodiments. Exemplary method 2600 may be implemented in systems such as system 100. As shown in the example of FIGS. 26A-26B, the matching may include bidding or booking of a unit or room in a residence of a care provider. The booking or bidding price may include unit base price+care level price. Each consumer may purchase an insurance-like instrument associated with their booking or bid. For bids, a consumer may bid on many residences within a time period, the highest bid amount will be held in escrow and the consumer may purchase an insurance-like instrument on that amount. Consumers may bid on multiple residences in the time period. The time period may be, for example, 24 hours, 48 hours, or 72 hours. Consumers may opt-out of a bid or booking at any time.

According to some embodiments, when a consumer registers with the system (such as system 100), the consumer may use all consumer functionalities of the system. The registered consumer may select and compare selected residences to make a well informed decision to book or bid. After booking or bidding, the registered consumer receives updates on process progress so that he/she can understand all the steps of the process. Registered consumers may bid on multiple residences. When the registered consumer bids, the consumer may provide payment information. For multiple bids, the highest bid amount is held in escrow. The registered consumer may see a status of their bids so that the registered consumer may decide which one to accept or deny. Each registered consumer may purchase an insurance-like instrument on their booking so that if they change their mind, the registered consumer won't lose all the money spent on the bid.

If for example, a provider has only one room that received several bids from different registered consumer, then as soon as the provider approves these bids and one of those registered consumers confirms the booking of that room before others, the system may automatically send bid declining notifications to the other registered customers that made a bid for that room.

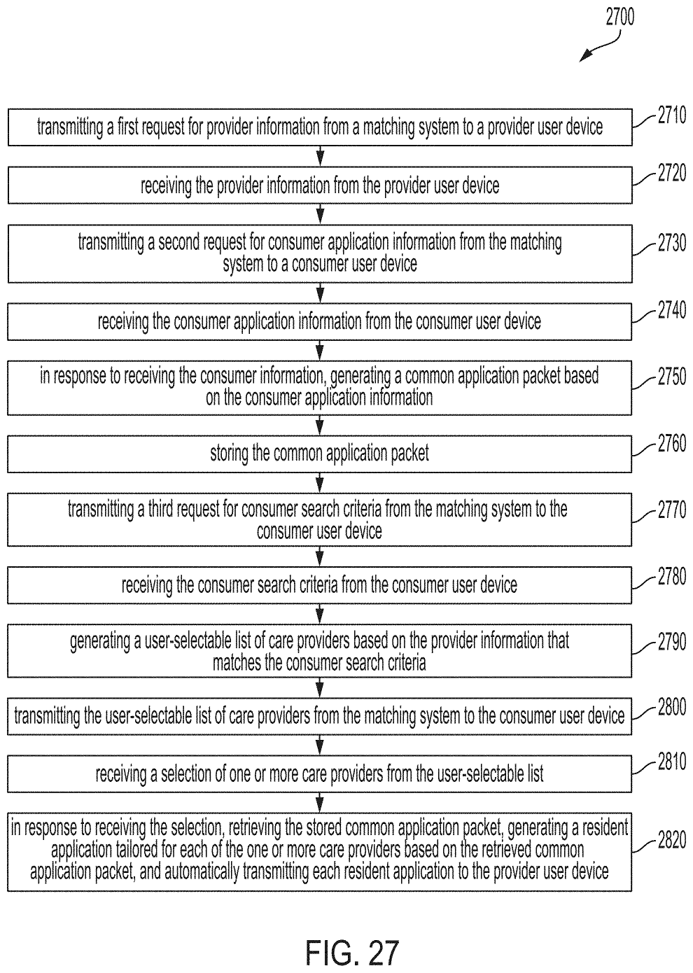

FIG. 27 shows a flowchart that describes an example method 2700 for consumers finding, bidding and booking care providers, according to some embodiments. Exemplary method 2700 may be implemented in systems such as system 100.

At step 2710, a first request for provider information from a matching system to a provider user device may be transmitted. According to some embodiments, the provider information may include information such as facilities, viewing pricing, services, photos, and reviews associated with the care facilities. According to some embodiments, examples of provider information of the first request are shown in FIGS. 15A-16H. According to some embodiments, the provider user device may be include a graphical user interface (such as interface 140) for displaying the first request. At step 2720, the provider information from the provider user device may be received.

At step 2730, a second request for consumer application information from the matching system to a consumer user device may be transmitted. According to some embodiments, the consumer application information may include details that care providers associated with the system typically request from consumers interested in becoming a resident at their provider facility. According to some embodiments, the consumer application information may include consumer demographic information, elements of their search criteria, consumer health information, and a level of care applicable for the consumer. According to some embodiments, the consumer application information may include one or more elements of the initial search information 206 shown in FIG. 1. According to some embodiments, the consumer user device may be include a graphical user interface (such as interface 120) for displaying the second request. At step 2740, the consumer application information from the consumer user device may be received.

At step 2750, in response to receiving the consumer information, a common application packet based on the consumer application information may be transmitted. According to some embodiments, the common application packet may include collected demographic information and documents required for resident intake process and allow the consumer to use the common application packet to apply to multiple care providers. At step 2760, storing the common application packet. In some embodiments, the common application data may be stored in a centralized database. In some embodiments, the matching system may be configured to store the common application packet until the system receives a user selection to send the common application packet to a selected care provider.

At step 2770, a third request for consumer search criteria from the matching system to the consumer user device may be transmitted. In some embodiments, the search criteria may include a preferred location, consumer's care needs, and price information. In some embodiments, the search criteria may include a price preference for one or more available rooms at a residence of the one or more care providers, and the available rooms have a minimum price set by the one or more care providers, and wherein the price preference equals or exceeds the minimum price. In some embodiments, the search criteria may include one or more elements of the initial search information 206 shown in FIG. 1. If a consumer's care needs (such as a level of care for the consumer) is not provided, the matching system may generate a recommendation of a level of care using a scoring process based on information received from the consumer. In some embodiments, the system may use method 2300 for generating a recommendation of a level of care. At step 2780, receiving the consumer search criteria from the consumer user device.

At step 2790, a user-selectable list of care providers based on the provider information that matches the consumer search criteria may be generated. According to some embodiments, the user-selectable list of care providers may be generated using method 2400. According to some embodiments, an example of a user-selectable list is shown in FIGS. 54-5F. At step 2800, the user-selectable list of care providers from the matching system to the consumer user device may be transmitted. According to some embodiments, the selection may include a specification of booking or bidding preferences. According to some embodiments, booking specification may include one or more of booking or bidding options, room preferences, level of care options, and additional services. The booking specifications may be displayed, for example, via informational page 400, room selection page 500, level of care selection page 600, additional services page 700. According to some embodiments, bidding specifications may include one or more of a price bid from the consumer, consumer contact information, room preferences, and level of care options. According to some embodiments, bidding specification may be displayed, for example, via offer page 1000.

At step 2810, a selection of one or more care providers from the user-selectable list may be received. In some embodiments, an example of confirmation that the selection has been received is shown in FIG. 11. At step 2820, in response to receiving the selection, the stored common application packet may be retrieved, a resident application tailored for each of the one or more care providers based on the retrieved common application packet may be generated, and each resident application may be automatically transmitted to the provider user device. According to some embodiments, booking options for a consumer stay at a residence of the one or more care providers may be automatically provided based on selection of the one or more care providers. According to some embodiments, resident application may be accessible to a care provider of the one or more care providers associated with each resident application via the provider user device. According to some embodiments, FIG. 14 shows an example of resident applications accessible to a care provider. According to some embodiments, administrative information associated with one or more of the resident applications may be transmitted from the matching system to the third user device and one or more administrative updates may be received by the matching system from the third user device. The administrative information may be associated with administrative tasks of an administrator. The administrative tasks, may include, for example, tasks regarding lead role assignments, bulk uploads, addition and deletion of residences and organizations from the matching system, updates to residence and organization information, account maintenance for consumers and providers. According to some embodiments, the administrative updates may be updates based on the administrative information. According to some embodiments, the administrative updates may be updates to the administrative information. According to some embodiments the administrative updates may include data changes on behalf of consumers, providers, or a combination thereof.

### Examples for Level of Care Assessment

Below are examples of questions of a preliminary level of care assessment, categorized by exemplary activities.

Bathing: Are you, or the person you are looking to get care for, able to bathe themselves in a bath or shower? Are you, or the person you are looking to get care for, able to stand in a shower independently for at least ten minutes? Are you, or the person you are looking to get care for, able to get out of a bathtub independently?

A series of bathing questions: Are you able to complete any part of the bathing process at all? Can you get in and out of the tub without assistance (machine or human)? Can you turn on the water, bathe entire body or take a sponge bath at the sink without assistance? Do you require immersion bathing? IF NO to any of the above, what type of help: Mechanical Only: Needs equipment or a device such as a shower, tub, chair, stool, grab bars, pedal or knee controlled faucet, long-handled brush and/or mechanical lift to complete the bathing process; Human Help Only (Supervision): prompting or verbal cues to safely complete washing entire body or need someone to teach them how to bathe; Human Help (Physical Assistance): Need someone to fill tub or bring water to the individual, need help getting into/out of tub or shower, help the individual towel dry; Mechanical & Human Help (Supervision): Need equipment or device and requires prompting/verbal cues to safely complete washing the entire body. Includes those who need someone to teach them how to bathe; Mechanical & Human Help (Physical Assistance): Needs equipment and physical assistance to complete task; and Performed by Others: Individual does not take part in the activity at all.

In some embodiments, if a response of “mechanical only” is received, the system may be configured to assign an indicator that describes the applicant as semi-dependent for this activity. If a response of “human help only (supervision)” is received, the system may be configured to assign an indicator that describes the applicant as semi-dependent for this activity. If a response of “human help (physical assistance)” is received, the system may be configured to assign an indicator that describes the applicant as dependent for this activity. If the only need for help is to help wash back and feet, then the system may be configured to describe the applicant as not needing help for this activity. If a response of “mechanical & human help (supervision)” is received, the system may be configured to assign an indicator that describes the applicant as dependent for this activity. If a response of “mechanical & human help (physical assistance)” is received, the system may be configured to assign an indicator that describes the applicant as dependent for this activity. If a response of “performed by other” is received, the system may be configured to assign an indicator that describes the applicant as being totally dependent for this activity.

Dressing: Are you, or the person you are looking to get care for, able to dress themselves independently? Are you, or the person you are looking to get care for, able to zip zippers or open/close buttons independently? Are you, or the person you are looking to get care for, able to tie shoe laces independently? Are you, or the person you are looking to get care for, able to perform basic grooming, such as combing hair, shaving, washing hands, etc.? Are you, or the person you are looking to get care for, able to perform basic grooming without reminders?

A series of dressing questions: Are you bedridden? If yes, the system may be configured to assign an indicator that describes the applicant as being totally dependent for this activity. If no, proceed with next question. If no, are you able to complete the dressing process without help from others or without the use of equipment or devices (excluding help tying shoes)? If yes, the system may be configured to assign an indicator that describes that the applicant does not need help. If No, choose between the following options: Mechanical Help Only: In order to complete the dressing process you need assistance from specific equipment or device such as a long-handled shoe horn, zipper pull, specially designed clothing, walker with an attached basket; Human Help Only (Supervision); Human Help Only (Physical Assistance); Mechanical+Human (Supervision); and Mechanical+Human (Physical Assistance).

In some embodiments, and similar to the bathing questions, if a response of “mechanical help only” is received, the system may be configured to assign an indicator that described the applicant as semi-dependent for this activity.

Toileting: Are you, or the person you are looking to get care for, capable of using the toilet without any assistance?

Transferring: Are you, or the person you are looking to get care for, capable of transferring themselves to and from bed, chair?

Eating/Feeding: Can you, or the person you are looking to get care for, eat or drink independently? Are you, or the person you are looking to get care for, able to maintain dietary restrictions independently? Are you, or the person you are looking to get care for, able to use basic utensils, such as a knife or fork, independently?

Continence—Bowel and Bladder: Can you, or the person you are looking to get care for, maintain continence?

Walking: Are you, or the person you are looking to get care for, able to walk independently for varying lengths of time? Are you, or the person you are looking to get care for, able to maintain a consistent walking speed independently? Are you, or the person you are looking to get care for, able to maintain a consistent gait independently?

Wheeling: Would you, or the person you are looking to get care for, be able to wheel themselves around (if they have a wheelchair)?

Mobility: Are you, or the person you are looking to get care for, independently mobile? Are you, or the person you are looking to get care for, able to evacuate themselves independently in case of emergency? Are you, or the person you are looking to get care for, able to balance themselves? Are you, or the person you are looking to get care for, able to arrange transportation for themselves? Are you, or the person you are looking to get care for, able to drive? Do you, or the person you are looking to get care for, require transportation to appointments or shopping?

Meal Preparation: Are you, or the person you are looking to get care for, able to prepare their own meals? Are you, or the person you are looking to get care for, able to maintain special diet restrictions independently?

Housekeeping: Are you, or the person you are looking to get care for, able to perform basic home maintenance, such as dusting, washing dishes, etc? Are you, or the person you are looking to get care for, able to make and receive phone calls independently? Are you, or the person you are looking to get care for, able to make appointments independently?

Laundry: Are you, or the person you are looking to get care for, able to fold or hang articles of clothing independently? Are you, or the person you are looking to get care for, able to operate a washer or dryer independently?

Money Management: Are you, or the person you are looking to get care for, able to budget independently? Are you, or the person you are looking to get care for, able to schedule payments independently? Are you, or the person you are looking to get care for, able to shop independently?

Medication Administration: Are you, or the person you are looking to get care for, able to self-administer medication independently? Are you, or the person you are looking to get care for, able to keep track of medications without reminders?

Memory Care: Are you, or the person you are looking to get care for, able to remember basic tasks, such as taking medication, grooming, etc? Are you, or the person you are looking to get care for, able to recall important items independently, such as birthdays, holidays, month/day/year? Are you, or the person you are looking to get care for, able to independently identify environmental needs and meet them? Are you, or the person you are looking to get care for, able to independently recognize friends or loved ones?

### Examples of User Stories

Consumer

In some embodiments, the system may be configured to allow a consumer to use the system to do one or more of the following: to search for residences that meet consumer needs, so that the consumer can ultimately choose one to reside in; to search residences by region, type of care, move-in date, price range, care level; to see results of acceptable elder care residences that meet consumer criteria, so that the consumer can ultimately live there (acceptable may mean affordable, specific wanted location, care needs met, residence safety indicators, residence layout); to further refine the search results based on more detailed criteria so that the consumer can get exactly what the consumer wants and needs (detailed criteria including location, price range, care type, care level, responsiveness, etc);

In some embodiments, the system may be configured to allow an unregistered consumer to use the system to do one or more of the following: to see the limited details of un-registered consumer selected residences that match the un-registered consumer's criteria to encourage the un-registered consumer to sign up and get more information about a specific residence(s) (the limited details may include no more than 3 photos of residence, lists 2 of the amenities, ratings with no more than 2 reviews, and link at the bottom urging registration to find out more about this residence and ability to compare others); to subscribe to updates on deals so that the un-registered consumer can see trends based on the un-registered consumer needs (Subscription is not registration; Subscription allows a consumer to specify their email address, phone, location, and the frequency that they receive emails or texts on Deals);

In some embodiments, the system may be configured to allow an unregistered consumer to use the system to register on the system so that registered user can use all of its functionalities and so that the consumer process to bid or book is easy and smooth.

In some embodiments, the system may be configured to allow a registered consumer to use the system to do one or more of the following: to add care team members, family members, share results, interface with a residence, collect documentation, apply, etc; to see all of the details of the consumer selected residence that matches consumer criteria so that the consumer can make an informed decision to bid or book (details may include residence reviews, staff pics, unit details, supported care levels, virtual tour, additional services, and their associated descriptions and costs, book and bid options, nearest hospitals); to use a simple questionnaire to assess a consumer care level; to share consumer search results with the people in their family and on their care team so that they can be in the know; to save favorite residences, so that the consumer can compile all search results and access at a later date; to get feedback from family members and care team of the search results that they like so that the consumer can include their input in their decision-making; to provide access rights to family members and care team (trusted agents—for example financial POAs or healthcare POAs—have the same rights as the consumer and view-only can only see shared results and progress); to select and compare favorite selected residences, so that the consumer can make a well informed decision to book or bid; after booking and/or bid, to receive updates on application progress so that the consumer can understand all the steps of the process; to bid on multiple residences, so that the consumer can have a chance at living in places that the consumer would not be able to afford otherwise; to provide payment information when the consumer places a bid (for multiple bids, the highest bid amount is held in escrow); to see bidding history on a room and or unit, so that the consumer can make informed decisions when the consumer bids on residences (bidding history may be provided for both the residences and a user's own history); to see the status of consumer bids so that the consumer can decide which one to accept or deny; to accept or reject a residence-approved bid so that the consumer can start the formal application process.

In some embodiments, the system may be configured to allow a registered consumer to use the system to do one or more of the following: to book a residence, so that the consumer can move in; to provide payment information during booking (booking amount is held in escrow); to share the residence the consumer booked or bid on with a care team and or family members so that they are in the know; to upload and save all the documentation needed for residence application so that the process can be smooth; to delegate the authority to upload documentation on their behalf so that the process can be smooth and stress-free; to send saved documentation to the residence once booking or bid is accepted, so that the process is efficient; to exchange messages with the residence that the consumer is applying to in order to get new requirements and ask questions; to schedule off-line application services so that they can move in (off-line application services may include, for example, TB tests, notarizations, conducting virtual consumer physical exams, scheduling in-person physical exams, scheduling an in-person tour); to create a review on a residence so that the consumer can share their experience; to provide a review of the system once the consumer is set to move in so that the consumer can help improve the system; to provide a 30-day review after move-in so that the consumer can provide updates on the process; to create a review on a staff member so that the consumer can share their experience; to purchase an insurance-like instrument on booking so that if the consumer changes their mind, all funds may not be lost (They may be refunded 80% of deposit. The 20% may be kept as a service fee); to purchase this insurance-like instrument on a booking so that if the consumer changes their mind, all funds may not be lost; to have the system recommend residences that meet the consumer needs, so that the consumer can ultimately choose one to reside in; to receive notifications on the registration status of the people that the consumer specified as being on my care team or family; to see a list of estate agents in the consumer's area so that the consumer can choose one; to see the list of referral agents in the consumer's area so that the consumer can choose one.

Caregiver

In some embodiments, the system may be configured to allow a caregiver to use the system to do one or more of the following: to search for residences that meet the consumer (or caree(s)) needs, so that the caregiver can ultimately choose a residence for the consumer to reside in; to search by region, type of care (long term vs. short term), move-in date, price range, care level, residence safety indicators, residence layout; to see the results of acceptable elder care residences that meet the criteria for each caree so that the caregiver can help them ultimately live in a residence and receive proper care (acceptable may include specific wanted region, type of care needs met, specific move-in date, affordability, care level, residence safety indicators, residence layout); to further refine the search results based on more detailed criteria so that the caregiver can get exactly what the carees want and need (detailed criteria may include location, price range, care type, care level, responsiveness, quality of care available to the caree); to subscribe for updates on deals so that the caregiver can see trends based on the caree(s) needs (Subscription is not registration. Subscription allows a caregiver to specify as much or as little in terms of filters (location, price range, care type, care level, responsiveness) as they need. It also allows them to specify the frequency that they receive emails or texts on deals); and to register on the system in order to use all of its functionalities so that the process is easy and smooth for the caregiver and the caree(s).

In some embodiments, the system may be configured to allow a registered caregiver to use the system to do one or more of the following: to add multiple carees, the family members of the carees, share results, interface with a residence, collect documentation, delegate authorization as necessary, and apply for multiple people (this includes residence reviews, staff pics, unit details, supported care levels, additional services, and their associated descriptions and costs, book and bid options, nearest hospitals); to view the profiles of those carees, so that the caregiver can bid, book, arrange appointments, and see progress for their carees; to share caregiver search results with the caree's family and their care team so that they can be in the know; to get feedback from the caree's family members and care team of the search results that they like so that the caregiver can include their input in the decision-making process; to select and compare selected residences, so that the caregiver can make a well informed decision for their caree(s); to receive updates on application progress so that the caregiver can rest easy knowing their caree(s) is progressing through necessary steps; to bid on residences, so that the caree(s) can have a chance at living in places they would not be able to afford otherwise (when the caregiver places a bid, the caregiver can themselves provide payment information, or the caregiver may designate another to provide payment information (bid amount held in escrow); to see bidding history, so that the caregiver can make informed decisions when the caregiver places a bid on residences for the caree(s) (bidding history for both the residences, and a user's own history); to book a residence, so that the caregiver can move the caree(s) in; to bid on multiple residences, so the caree(s) can have a chance at living in places the caree(s) wouldn't be able to afford otherwise (for multiple bids, the highest bid amount may be held in escrow); to see the status of the caregiver's bids so that the caregiver can help decide which one to accept or deny; to accept or reject a residence-approved bid so that the caregiver can help start the formal application process; to see progress updates on the formal application process for the caree so that the caregiver can assist as necessary; to share the residence that the caregiver booked or bid on with the caree(s), their care team and or family members so that they are in the know; to upload and save all the documentation needed for the residence application for the caree so that the process can be smooth; to delegate the authority to upload documentation on behalf of the caree(s) so that the process can be smooth and stress-free; to send saved documentation to the residence once the booking or bid is accepted, so that the process is efficient; to exchange messages with the residence(s) that the caregiver is applying to for the caree(s) in order to ask and/or answer questions; to specify the cadence at which the caregiver receives notifications on the updates from the system so that the caregiver can act in a timely manner and avoid things expiring (cadence options are time-based or trigger-based); to schedule off-line application services so that the caree's application can proceed through necessary steps to get to move in (off-line services may include, for example, TB tests, notarizations, scheduling physical exam, scheduling an in-person tour); to find local resources, so that the caregiver can get support in this process; to find all the people, and steps in order to make the process efficient; to find support or services that can help ease their application, care assessment, and move-in.

Referral Agent

In some embodiments, the system may be configured to allow a referral agent to use the system to do one or more of the following: to provide evidence/proof/documentation of a referral agent status so that the referral agent can access the system; to upload referral agent credentials electronically to the system for verification; to quickly and seamlessly process referral information electronically so that the referral agent can start referring people as quickly as possible; to provide information on a company associated with the referral agent, and a method that the referral agent would want funds disbursed; to agree to a referral contract so that the parties involved have clear agreements, and expectations; to see how much money the referral agent has earned on the system so that the referral agent can refine their referral marketing and operations with the system (this includes the history of referred carees; highlighting the ones that use the referral agent's selection); to see the referred carees that selected the options that the referral agent provided with updates on the dashboard (applied, declined, moved-in) so that the referral agent can know a timeline of when payment may be disbursed to the referral agent; to share results with a caree or caregiver so that they can register with the system to see the results (includes ability to show rated results to caree or caregiver); to provide details of a caree or caregiver so that they can register with the system and see the search results that the referral agent found; and to know the cost of room, care, and additional services for clients of the referral agent so that the referral agent can provide their clients with the most relevant information in order to help place them.

Estate Planner

In some embodiments, the system may be configured to allow an estate planner to use the system to do one or more of the following: to search for residences that meet an estate planner's caree(s) needs, so that the estate planner can ultimately choose a residence for them; to search by region, type of care, move-in date, price range, care level; to see results of acceptable elder care residences that meet the criteria for each caree, so that the estate planner can help them ultimately live at a residence and receive proper care (acceptable includes affordable, specific wanted location, care needs met, residence safety indicators, residence layout); to further refine the search results based on more detailed criteria so that the estate planner can get exactly what carees want and need (details may include location, price range, care type, care level, responsiveness, etc); to subscribe to updates on deals so that the estate planner can see trends based on the needs of the caree(s) (Subscription is not registration. Subscription allows an Estate Planner to specify as much or as little in terms of filters (location, price range, care type, care level, responsiveness) as they need. It also allows the estate planner to specify the frequency that they receive emails or texts on Deals); to register with the system in order to use all of its functionalities so that the process is easy and smooth for my carees and the estate planner.

In some embodiments, the system may be configured to allow an estate planner registered with the system to use the system to do one or more of the following: to add multiple carees, the family members of the caree(s), share results, interface with a residence on behalf of their caree(s), collect documentation, delegate authorization as necessary, and apply for multiple people (this includes residence reviews, staff pics, unit details, supported care levels, additional services, and their associated descriptions and costs, book and bid options, nearest hospitals); to switch through the profiles of the caree(s), so that the estate planner can bid, book, arrange appointments, and see progress for the caree(s) (this includes an ability to switch through multiple caree profiles); to share search results with the caree's family and their care team so that they can be in the know; to get feedback from caree's family members and care team of the search results that they like so that the estate planner can include their input in decision-making; to select and compare residences, so that the estate planner can help make a well informed decision for the caree(s); to receive updates on application progress so that the estate planner can know whether the caree is progressing through necessary steps; to bid on residences, so that the caree(s) can have a chance at living in places they wouldn't be able to afford otherwise. (When the estate planner places a bid, the estate planner can themselves provide payment information (if the caree has designated the estate planner to do so on his or her behalf), or delegate another to provide payment information. Bid amount is held in escrow); to see bidding history, so that the estate planner can make informed decisions when placing a bid on residences for the caree(s). (Bidding history for both the residences, and a user's own history); to book a residence, so that the estate planner can move the caree(s) in. (When the estate planner books a residence, the estate planner themselves may provide payment information (if the caree has designated the estate planner to do so on his or her behalf), or may delegate another to provide payment information. (Booking amount is held in escrow); to see progress updates on the application for caree(s) so that the estate planner can assist as necessary; to share the residence that the estate planner has booked or bid on with the caree(s), their care team and or family members so that they are in the know; to upload and save all the documentation needed for the residence application for the caree so that the process can be smooth; to delegate the authority to upload documentation on the caree's behalf so that the process can be smooth and stress-free; to send saved documentation to the residence that the estate planner is applying to so that the process is efficient; to exchange messages with the residence that the estate planner is applying to in order to ask and answer questions; to schedule off-line application services so that the caree(s) can move in (off-line application services may include, for example, TB tests, notarizations, physical exams through a telehealth platform, etc.); to find local resources, so that the estate planner can get support in this process; to find the people, and steps in order to make the process efficient; to find support or services that can help ease their application, care assessment, and move-in process.

Provider

The system is configured to allow a provider to use the system to do one or more of the following: to enter details (includes care level, staff, amenities, pictures, virtual tour, etc) about a residence (facility) so that it is listed in the system; to enter the approval flow for provider staff's actions on the system for supervisional visibility and control; to create listings easily for allotting more time on business matters versus inventory management and marketing; to view the listed residences for aggregating data from all residences; to view listings of each residence; to know how much revenue the provider is making using the system for optimizing provider income; to electronically specify the documentation required for a residence so that the process can be streamlined; to electronically request the documentation required for a residence so that the process can be streamlined; to electronically receive the documentation required for each residence so that the process can be streamlined; to electronically provide feedback on the requested documents to acknowledge the consumers progress during the search, application, and move-in process; to receive a notification that there is a document that is awaiting approval by the provider; to track the stage that an application is in for moving prospective clients swiftly through the process; to see the cost, services offered, occupancy and accommodations in the current marketplace, for assessing if offerings by the provider should be adjusted; to formally track the movement of a caree within and outside of the residence for soliciting feedback and improving provider operations (movement includes caree leaving residence, caree moving to a different unit or to a different level of Care inside residence, caree passing on); to respond to user reviews for addressing feedback and improving.

Residence Provider

The system is configured to allow a residence provider to use the system to do one or more of the following: to see the applications to the residence; to see messages sent to them by prospective residents; to see the residents that used the system to move in to track the system efficiency; to approve or deny applicants who have submitted their application and supporting documentation for managing residence occupancy and staff; to organize past applications, declines, archive and download for analysis at a later date; to send applications completed to cold storage, only log transaction data and only show provider the transaction log.

Organization Provider

The system is configured to allow an organization provider to use the system to do one or more of the following: to send messages to residence managers for collaborating on issues, standards, and business matters; to receive messages from residence managers for collaborating on issues, standards, and business matters; to identify specific residences that are at either end of the performance spectrum in order to improve operations at under-performing locations or benchmark operations at higher-performing locations; know what locations have higher demands to inform opportunities of new construction, evaluate buying options to increase supply, or optimize use of the bidding system.

Administrator

The system is configured to allow an administrator to use the system to do one or more of the following administrative tasks: to assign the lead role for an organization so that any major system decisions or changes go through an authorization process; to assign the lead role for a residence so that the administrator can delegate tasks; to upload organization details in bulk so that the administrator can make the onboarding process easier for organizations; to upload residence details in bulk so that the administrator can make the onboarding process easier for residences; make access easier for organizations; to upload unit details in bulk so that the administrator can make the onboarding process easier for organizations; to upload room details in bulk so that the administrator can make the onboarding process easier; to be able to delete residences and organizations from the platform; to change information for residences and organizations in the system; and to put a residence or organization's accounts on hold so that they can have the opportunity to rectify a situation. According to some embodiments, an administrator user device may be configured to display a graphical user interface (such as interface 160). The administrator user device may be accessible to a system administrator (such as administrator 115). According to some embodiments, administrative information may be information associated with one or more the administrative tasks. According to some embodiments, the matching system (such as system 100) may be configured to collect administrative information from one or more of the consumer user device, the provider user device, and the matching system, and transmit the administrative information to the administrator user device. According to some embodiments, the administrator user device may be configured to receive administrative updates from a system administrator and transmit the administrative updates to the matching system. The matching system may be configured to receive the administrative updates from the administrator user device and implement the administrative changes to the matching system. According to some embodiments, the administrative changes may include data changes on behalf of consumers, providers, or a combination thereof. According to some embodiments, the administrative information may be associated with one or more resident applications or the resident application process of one or more resident applications.

### EMBODIMENTS

Embodiment 1: A method for recommending a level of care for consumers, comprising:

transmitting a first request for consumer information from the matching system to a consumer user device, the consumer information comprising a plurality of independency levels of a consumer, each independency level of the plurality of independency levels represents the consumer's ability to complete an activity of a plurality of activities, each independency level is associated with a designated point value;

receiving the consumer information from the consumer user device;

determining whether one or more of the designated point values associated with independency levels that represent the consumer's ability to complete one or more activities of the plurality of activities lie within a threshold range,

calculating a mathematical result based on the one or more designated point values and whether the one or more of the designated point values lie within the threshold range;

generating a recommended level based on the mathematical result; and

transmitting the recommended level of care to the consumer user device.

Embodiment 2: The method of embodiment 1, comprising transmitting a second request for provider information from the matching system to a provider user device; receiving the provider information from the provider user device; transmitting a third request for search criteria from the matching system to the consumer user device; and receiving the search criteria from the consumer user device.

Embodiment 3: The method of embodiment 2, comprising generating a user-selectable list of care providers based on the provider information that match search criteria and offers the recommended level of care; transmitting the user-selectable list from the matching system to the consumer user device; receiving a selection of one or more care providers from the user-selectable list; and in response to receiving the selection, automatically providing booking options for a consumer stay at a residence of the one or more care providers.

Embodiment 4: The method of embodiment 3, wherein the search criteria comprises a price preference for one or more available rooms at the residence of the one or more care providers, and the available rooms have a minimum price set by the one or more care providers, and wherein the price preference equals or exceeds the minimum price.

Embodiment 5: The method of embodiment 1, wherein the one or more activities is associated with memory care.

Embodiment 6: The method of embodiment 1, wherein the plurality of care options include one or more of memory care, independent living, assisted living, and skilled nursing.

Embodiment 7: A method for matching consumers to care providers, comprising:

transmitting a first request for provider information from a matching system to a provider user device;

receiving the provider information from the provider user device;

transmitting a second request for search criteria from the matching system to a consumer user device, the search criteria comprising at least one or more of a preferred location, price information, and consumer care needs;

receiving the search criteria from the consumer user device;

in response to receiving the search criteria, determining a first set of coordinates based on the search criteria; and

identifying first care providers based on the provider information that have available rooms that satisfy the search criteria and are located in a first area associated with the first set of coordinates.

Embodiment 8: The method of embodiment 7, comprising counting an amount of the first care providers; if the amount of first care providers is at least one, determining whether the amount of the first care providers is equal to, above, or less than a minimum amount; and if it is determined that the amount of the first care providers is equal to or above the minimum amount, automatically transmitting the first care providers as part of a list of care providers from the matching system to the consumer user device.

Embodiment 9: The method of embodiment 8, wherein if the amount of first care providers is not at least one, updating the first set of coordinates to equal a second set of coordinates, the second set of coordinates having a second area that is larger than the first area by a first percentage.

Embodiment 10: The method of embodiment 8, wherein if it is determined that the amount of first care providers is less than the minimum amount, identifying second care providers that have available rooms that satisfy the search criteria and that are located in a third area associated with a third set of coordinates, the third area having a value larger than the second area by a second percentage, counting an amount of the second care providers, and updating the first set of coordinates to equal the third set of coordinates if the amount of second care providers is greater than the amount of first care providers by over a third percentage.

Embodiment 11: The method of embodiment 10, wherein if the amount of second care providers is not greater than the amount of first care providers by over the third percentage, detecting a center of a grouping of the second care providers, the center having center coordinates, setting the center coordinates as a center of a fourth area, the fourth area associated with a fourth set of coordinates, and identifying fourth care providers that have available rooms that satisfy the search criteria and that are located in the fourth area.

Embodiment 12: The method of embodiment 11, wherein, if an amount of fourth care providers is greater than the amount of third providers by over a fourth percentage, updating the first set of coordinates to equal the fourth set of coordinates.

Embodiment 13: The method of embodiment 12, wherein the fourth percentage is equal to the third percentage and the second percentage is equal to the first percentage.

Embodiment 14: The method of embodiment 11, wherein if an amount of fourth care providers is not greater than the amount of third providers by over a fourth percentage, transmitting the third care providers as part of the list of care providers from the matching system to the consumer user device.

Embodiment 15: A method for matching consumers to care providers comprising:

transmitting a first request for provider information from a matching system to a provider user device;

receiving the provider information from the provider user device;

transmitting a second request for consumer application information from the matching system to a consumer user device;

receiving the consumer application information from the consumer user device;

in response to receiving the consumer information, generating a common application packet based on the consumer application information;

storing the common application packet;

transmitting a third request for consumer search criteria from the matching system to the consumer user device;

receiving the consumer search criteria from the consumer user device;

generating a user-selectable list of care providers based on the provider information that matches the consumer search criteria;

transmitting the user-selectable list of care providers from the matching system to the consumer user device;

receiving a selection of one or more care providers of the user-selectable list; and

in response to receiving the selection, retrieving the stored common application packet, generating a resident application tailored for each of the one or more care providers based on the retrieved common application packet, and automatically transmitting each resident application to the provider user device.

Embodiment 16: The method of embodiment 15, wherein each resident application is accessible to a care provider of the one or more care providers associated with each resident application via the provider user device.

Embodiment 17: The method of embodiment 15, wherein the consumer application information comprises one or more of demographic information, consumer health information, and a level of care applicable for the consumer.

Embodiment 18: A system for matching consumers to care providers, comprising:

a first user device configured for registered providers;

a second user device configured for consumers;

a third user device configured for system administrators;

a matching system configured to communicatively connected the first user device, the second user device, and the third user device, the matching system comprising one or more programs that include instructions to:
transmit a first request for provider information to the first user device;
receive the provider information from the first user device;
transmit a second request for consumer application information to the second user device;
receive the consumer application information from the second user device;
in response to receiving the consumer information, generate a common application packet based on the consumer application information;
store the common application packet;
transmit a third request for consumer search criteria from the matching system to the second user device;
receive the consumer search criteria from the second user device;
generate a user-selectable list of care providers based on the provider information that matches the consumer search criteria;
transmit the user-selectable list of care providers to the second user device;
receive a selection of one or more care providers of the user-selectable list; and
in response to receiving the selection, retrieve the stored common application packet, generate a resident application tailored for each of the one or more care providers based on the retrieved common application packet, and automatically transmit each resident application to the first user device.

Embodiment 19: The system of embodiment 18, wherein each resident application is accessible to a care provider of the one or more care providers associated with each resident application via the first user device, and wherein the one or more programs of the matching system includes instructions to transmit administrative information associated with one or more resident applications to the third user device and to receive one or more administrative updates from the third user device based on the administrative information.

Embodiment 20: The system of embodiment 18, wherein the consumer application information comprises one or more of demographic information, consumer health information, and a level of care applicable for the consumer.

Embodiment 21: A system for recommending a level of care for consumers, comprising:

a first user device configured for consumers;

a matching system communicatively connected to the first user device, the matching system comprising one or more programs that include instructions to:
transmit a first request for consumer information from the matching system to a consumer user device, the consumer information comprising a plurality of independency levels of a consumer, each independency level of the plurality of independency levels represents the consumer's ability to complete an activity of a plurality of activities, each independency level is associated with a designated point value;
receive the consumer information from the first user device;
determine based on the consumer information whether one or more of the designated point values associated with independency levels that represent the consumer's ability to complete one or more activities of the plurality of activities lie within a threshold range;
calculate a mathematical result based on the one or more designated point values and whether the one or more of the designated point values lie within the threshold range;
generate a recommended level based on the mathematical result; and
transmit the recommended level of care to the first user device.

Embodiment 22: The system of embodiment 21, comprising a second user device configured for registered providers, a third user device configured for system administrators, wherein the matching system is configured to communicatively connect the first user device, the second user device, and the third user device, and the one or more programs including instructions to: transmit a second request for provider information from the matching system to the second user device; receive the provider information from the second user device; transmit a third request for search criteria from the matching system to the first user device; and receive the search criteria from the first user device.

Embodiment 23: The system of embodiment 22, wherein the one or more programs including instructions to generate a user-selectable list of care providers based on the provider information that match search criteria and offers the recommended level of care; transmit the user-selectable list from the matching system to the consumer user device; receive a selection of one or more care providers of the user-selectable list; and in response to receiving the selection, automatically provide booking options for a consumer stay at a residence of the one or more care providers.

Embodiment 24: The system of embodiment 23, wherein the search criteria comprises a price preference for one or more available rooms at the residence of the one or more care providers, and the available rooms have a minimum price set by the one or more care providers, and wherein the price preference equals or exceeds the minimum price.

Embodiment 25: The system of embodiment 21, wherein the one or more activities is associated with memory care.

Embodiment 26: The system of embodiment 21, wherein the plurality of care options include one or more of memory care, independent living, assisted living, and skilled nursing.

Embodiment 27: A system for matching consumers to care providers, comprising:

a first user device configured for registered providers;

a second user device configured for consumers;

a third user device configured for system administrators;

a matching system configured to communicatively connect the first user device, the second user device, and the third user device, the matching system comprising one or more programs that include instructions to:
transmit a first request for provider information from the matching system to the first user device;
receive the provider information from the first user device;
transmit a second request for search criteria from the matching system to the second user device, the search criteria comprising at least one or more of a preferred location, price information, and consumer care needs;
receive the search criteria from the second user device;
in response to receiving the search criteria, determine a first set of coordinates based on the search criteria; and
identify first care providers based on the provider information that have available rooms that satisfy the search criteria and are located in a first area associated with the first set of coordinates.

Embodiment 28: The system of embodiment 27, wherein the one or more programs that include instructions to count an amount of the first care providers; if the amount of first care providers is at least one, determine whether the amount of the first care providers equal to, above, or less than a minimum amount; and if it is determined that the amount of the first care providers is equal to or above the minimum amount, automatically transmit the first care providers as part of a list of care providers from the matching system to the second user device.

Embodiment 29: The system of embodiment 28, wherein if the amount of first care providers is not at least one, update the first set of coordinates to equal a second set of coordinates, the second set of coordinates having a second area that is larger than the first area by a first percentage.

Embodiment 30: The system of embodiment 28, wherein if it is determined that the amount of first care providers is less than the minimum amount, identify second care providers that have available rooms that satisfy the search criteria and that are located in a third area associated with a third set of coordinates, the third area having a value larger than the second area by a second percentage, count an amount of the second care providers, and update the first set of coordinates to equal the third set of coordinates if the amount of second care providers is greater than the amount of first care providers by over a third percentage.

Embodiment 31: The system of embodiment 30, wherein if the amount of second care providers is not greater than the amount of first care providers by over the third percentage, detect a center of a grouping of the second care providers, the center having center coordinates, set the center coordinates as a center of a fourth area, the fourth area associated with a fourth set of coordinates, and identify fourth care providers that have available rooms that satisfy the search criteria and that are located in the fourth area.

Embodiment 32: The system of embodiment 31, wherein, if an amount of fourth care providers is greater than the amount of third providers by over a fourth percentage, update the first set of coordinates to equal the fourth set of coordinates.

Embodiment 33: The system of embodiment 32, wherein the fourth percentage is equal to the third percentage and the second percentage is equal to the first percentage.

Embodiment 34: The system of embodiment 31, wherein if an amount of fourth care providers is not greater than the amount of third providers by over a fourth percentage, transmit the third care providers as part of the list of care providers from the matching system to the second user device.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A method for recommending a level of care for consumers, comprising:
transmitting a first request for consumer information from the matching system to a consumer user device, the consumer information comprising a plurality of independency levels of a consumer, each independency level of the plurality of independency levels represents the consumer's ability to complete an activity of a plurality of activities, each independency level is associated with a designated point value;
receiving the consumer information from the consumer user device;
determining whether one or more of the designated point values associated with independency levels that represent the consumer's ability to complete one or more activities of the plurality of activities lie within a threshold range,
calculating a mathematical result based on the one or more designated point values and whether the one or more of the designated point values lie within the threshold range;
generating a recommended level based on the mathematical result; and
transmitting the recommended level of care to the consumer user device.

2. The method of claim 1, comprising transmitting a second request for provider information from the matching system to a provider user device; receiving the provider information from the provider user device; transmitting a third request for search criteria from the matching system to the consumer user device; and receiving the search criteria from the consumer user device.

3. The method of claim 2, comprising generating a user-selectable list of care providers based on the provider information that match search criteria and offers the recommended level of care; transmitting the user-selectable list from the matching system to the consumer user device; receiving a selection of one or more care providers from the user-selectable list; and in response to receiving the selection, automatically providing booking options for a consumer stay at a residence of the one or more care providers.

4. The method of claim 3, wherein the search criteria comprises a price preference for one or more available rooms at the residence of the one or more care providers, and the available rooms have a minimum price set by the one or more care providers, and wherein the price preference equals or exceeds the minimum price.

5. The method of claim 1, wherein the one or more activities is associated with memory care.

6. The method of claim 1, wherein the recommended level of care is based on a plurality of care options that include one or more of memory care, independent living, assisted living, and skilled nursing.

7. A method for matching consumers to care providers, comprising:
transmitting a first request for provider information from a matching system to a provider user device;
receiving the provider information from the provider user device;
transmitting a second request for search criteria from the matching system to a consumer user device, the search criteria comprising at least one or more of a preferred location, price information, and consumer care needs;
receiving the search criteria from the consumer user device;
in response to receiving the search criteria, determining a first set of coordinates based on the search criteria; and
identifying first care providers based on the provider information that have available rooms that satisfy the search criteria and are located in a first area associated with the first set of coordinates.

8. The method of claim 7, comprising counting an amount of the first care providers; if the amount of first care providers is at least one, determining whether the amount of the first care providers is equal to, above, or less than a minimum amount; and if it is determined that the amount of the first care providers is equal to or above the minimum amount, automatically transmitting the first care providers as part of a list of care providers from the matching system to the consumer user device.

9. The method of claim 8, wherein if the amount of first care providers is not at least one, updating the first set of coordinates to equal a second set of coordinates, the second set of coordinates having a second area that is larger than the first area by a first percentage.

10. The method of claim 8, wherein if it is determined that the amount of first care providers is less than the minimum amount, identifying second care providers that have available rooms that satisfy the search criteria and that are located in a third area associated with a third set of coordinates, the third area having a value larger than the second area by a second percentage, counting an amount of the second care providers, and updating the first set of coordinates to equal the third set of coordinates if the amount of second care providers is greater than the amount of first care providers by over a third percentage.

11. The method of claim 10, wherein if the amount of second care providers is not greater than the amount of first care providers by over the third percentage, detecting a center of a grouping of the second care providers, the center having center coordinates, setting the center coordinates as a center of a fourth area, the fourth area associated with a fourth set of coordinates, and identifying fourth care providers that have available rooms that satisfy the search criteria and that are located in the fourth area.

12. The method of claim 11, wherein, if an amount of fourth care providers is greater than the amount of third providers by over a fourth percentage, updating the first set of coordinates to equal the fourth set of coordinates.

13. The method of claim 12, wherein the fourth percentage is equal to the third percentage and the second percentage is equal to the first percentage.

14. The method of claim 11, wherein if an amount of fourth care providers is not greater than the amount of third providers by over a fourth percentage, transmitting the third care providers as part of the list of care providers from the matching system to the consumer user device.

15. A method for matching consumers to care providers comprising:
transmitting a first request for provider information from a matching system to a provider user device;
receiving the provider information from the provider user device;
transmitting a second request for consumer application information from the matching system to a consumer user device;
receiving the consumer application information from the consumer user device;
in response to receiving the consumer information, generating a common application packet based on the consumer application information;
storing the common application packet;
transmitting a third request for consumer search criteria from the matching system to the consumer user device;
receiving the consumer search criteria from the consumer user device;
generating a user-selectable list of care providers based on the provider information that matches the consumer search criteria;
transmitting the user-selectable list of care providers from the matching system to the consumer user device;
receiving a selection of one or more care providers of the user-selectable list; and
in response to receiving the selection, retrieving the stored common application packet, generating a resident application tailored for each of the one or more care providers based on the retrieved common application packet, and automatically transmitting each resident application to the provider user device.

16. The method of claim 15, wherein each resident application is accessible to a care provider of the one or more care providers associated with each resident application via the provider user device.

17. The method of claim 15, wherein the consumer application information comprises one or more of demographic information, consumer health information, and a level of care applicable for the consumer.

18. A system for matching consumers to care providers, comprising:
a first user device configured for registered providers;
a second user device configured for consumers;
a third user device configured for system administrators;
a matching system configured to communicatively connected the first user device, the second user device, and the third user device, the matching system comprising one or more programs that include instructions to:
transmit a first request for provider information to the first user device;
receive the provider information from the first user device;
transmit a second request for consumer application information to the second user device;
receive the consumer application information from the second user device;
in response to receiving the consumer information, generate a common application packet based on the consumer application information;
store the common application packet;
transmit a third request for consumer search criteria from the matching system to the second user device;
receive the consumer search criteria from the second user device;
generate a user-selectable list of care providers based on the provider information that matches the consumer search criteria;
transmit the user-selectable list of care providers to the second user device;
receive a selection of one or more care providers of the user-selectable list; and
in response to receiving the selection, retrieve the stored common application packet, generate a resident application tailored for each of the one or more care providers based on the retrieved common application packet, and automatically transmit each resident application to the first user device.

19. The system of claim 18, wherein each resident application is accessible to a care provider of the one or more care providers associated with each resident application via the first user device, and wherein the one or more programs of the matching system includes instructions to transmit administrative information associated with one or more resident applications from the matching system to the third user device and to receive one or more administrative updates from the third user device based on the administrative information.

20. The system of claim 18, wherein the consumer application information comprises one or more of demographic information, consumer health information, and a level of care applicable for the consumer.
